(19) United States
(12) Patent Application Publication
(10) Pub. No.: US 2016/0040185 A1
(43) Pub. Date: Feb. 11, 2016

(54) EXPRESSION VECTOR AND METHOD OF PREPARING A POLYPEPTIDE OF INTEREST USING THE SAME

(71) Applicant: Samsung Electronics Co., Ltd., Suwon-si (KR)

(72) Inventors: Su Jeong HWANG, Suwon-si (KR); Min-Kyung KIM, Seoul (KR); Sunkyu KIM, Suwon-si (KR); Chan moo LEE, Gyeonggi-do (KR)

(21) Appl. No.: 14/823,861

(22) Filed: Aug. 11, 2015

(30) Foreign Application Priority Data
Aug. 11, 2014 (KR) 10-2014-0103609

(51) Int. Cl. C12N 15/85; C07K 16/00

(52) U.S. Cl. CPC C12N 15/85; C07K 16/00; C12N 2800/107; C12N 2830/42; C07K 2317/56; C07K 2317/51

(57) ABSTRACT

A fusion polynucleotide including a human CMV promoter and an intron, a recombinant vector including the fusion polynucleotide and a gene encoding a polypeptide of interest, a recombinant cell comprising the recombinant vector, and a method of producing a polypeptide of interest using the recombinant vector or recombinant cell.

### CROSS-REFERENCE TO RELATED APPLICATIONS

This application claims the benefit of Korean Patent Application No. 10-2014-0103609 filed on Aug. 11, 2014 in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted herewith and identified as follows: 148,382 byte ASCII (Text) file named “721063_ST25.TXT” created Aug. 10, 2015.

### BACKGROUND OF THE INVENTION

1. Field

Provided is a fusion polynucleotide including a promoter and an intron, a recombinant vector including the fusion polynucleotide and a gene encoding a polypeptide of interest, a recombinant cell including the recombinant vector, and a method of producing a polypeptide of interest using the recombinant vector and/or the recombinant cell.

2. Description of the Related Art

Therapeutic proteins, such as antibodies, have emerged in the medical industry and have been developed as medicines for various targets. To commercialize and examine the effects of the developed therapeutic proteins, it is necessary to produce the proteins on a large scale.

The use of an animal cell to produce a therapeutic protein can increase the efficacy of the therapeutic protein compared to using microorganisms to produce a protein; however, animal cells are limited in that the amount of produced protein is small. To solve this problem, it is necessary to develop a recombinant vector capable of increasing protein productivity in an animal cell. This invention provides such vector.

### BRIEF SUMMARY OF THE INVENTION

An embodiment provides a fusion polynucleotide including a promoter and an intron. The fusion polynucleotide may be useful as a promoter, for example, a promoter operable in an animal cell (e.g., a mammalian cell).

Another embodiment provides a recombinant vector including the fusion polynucleotide including a promoter and an intron. The recombinant vector may be useful as an expression vector capable of expressing a polypeptide of interest in a host cell, when a gene encoding the polypeptide of interest is operatively linked therein. The recombinant vector may be one capable of being expressed in a host cell.

Another embodiment provides a recombinant vector including a gene encoding a polypeptide of interest and a fusion polynucleotide including a promoter and an intron, wherein the fusion polynucleotide is operatively linked to the gene encoding a polypeptide of interest. The recombinant vector may be one capable of being expressed in a host cell.

Another embodiment provides a recombinant cell including the recombinant vector, and provides a method for producing a polypeptide of interest using the recombinant vector or the recombinant cell.

### BRIEF DESCRIPTION OF THE DRAWINGS

FIG. 1 is a cleavage map of a vector comprising a fusion polypeptide comprising human CMV promoter and intron.

FIG. 2 is a graph showing an antibody production ratio when an anti-c-Met antibody is expressed using a recombinant vector comprising a fusion polypeptide of human CMV promoter and intron in 293F cells (CMV-Opti301: human CMV promoter only, CA-Opti301: human CMV promoter+Intron A, CK-Opti301: human CMV promoter+IGKV Intron, CC-Opti301: human CMV promoter+chimeric intron).

FIG. 3 is a graph showing an antibody production ratio when an anti-c-Met antibody is expressed using a recombinant vector comprising a fusion polypeptide of human CMV promoter and intron in CHO cells (hCMV-Opti301: human CMV promoter only, CA-Opti301: human CMV promoter+Intron A, hCK-Opti301: human CMV promoter+IGKV Intron, hCH-Opti301: human CMV promoter+IGH Intron, hCC-Opti301: human CMV promoter+chimeric intron).

FIG. 4 shows a sequence alignment result between SEQ ID NO: 116 and SEQ ID NO: 117, wherein the mutated residues of SEQ ID NO: 117 are outlined.

FIG. 5 shows a sequence alignment result between SEQ ID NO: 116 and SEQ ID NO: 118, wherein the mutated residues of SEQ ID NO: 118 are outlined.

FIG. 6 shows a sequence alignment result between SEQ ID NO: 116 and SEQ ID NO: 119.

FIG. 7 shows a sequence alignment result between SEQ ID NO: 116 and SEQ ID NO: 120.

FIG. 8 shows a sequence alignment result between SEQ ID NO: 116 and SEQ ID NO: 121.

FIG. 9 shows a sequence alignment result between SEQ ID NO: 116 and SEQ ID NO: 122, wherein the mutated residues of SEQ ID NO: 122 are outlined.

FIG. 10 shows a sequence alignment result between SEQ ID NO: 116 and SEQ ID NO: 123 (added residues are not shown).

FIG. 11 shows a sequence alignment result between SEQ ID NO: 116 and SEQ ID NO: 124, wherein the mutated residues of SEQ ID NO: 124 are outlined.

FIG. 12 shows an example of a cleavage map of a recombinant vector comprising a fusion polypeptide of human CMV promoter and intron.

FIG. 13 shows an example of a cleavage map of a recombinant vector comprising a fusion polypeptide of human CMV promoter and intron, a selection marker (GS or DHFR), and IRES (Internal Ribosome Entry Site).

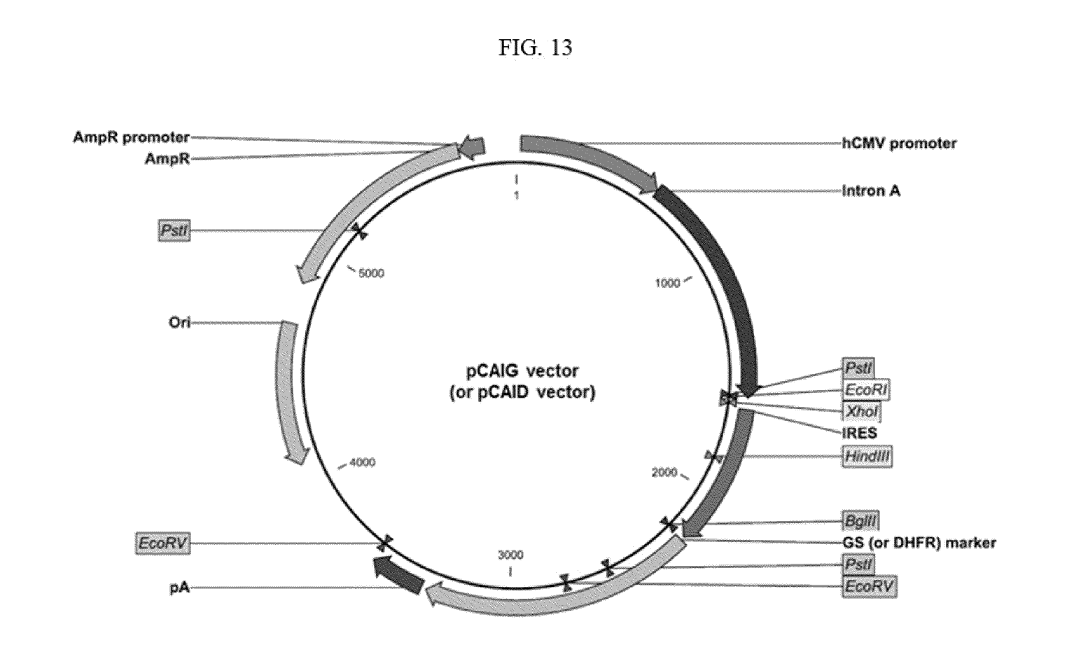

### DETAILED DESCRIPTION OF THE INVENTION

This disclosure relates to a use of an intron in preparing a recombinant vector for producing a polypeptide of interest.

An embodiment provides a fusion polynucleotide comprising a promoter and an intron. The fusion polynucleotide may be useful as a promoter, for example, capable of operating in an animal cell (e.g., a mammalian cell). Therefore, another embodiment provides a fusion promoter comprising a human CMV promoter and an intron.

A promoter is a transcription control factor (polynucleotide fragment) regulating the initiation of transcription of a gene, and generally has a length of about 100 to about 2000 bp, about 100 to about 1500 bp, or about 100 to about 1000 bp.

In this disclosure, any promoter capable of regulating the initiation of transcription of a gene in a cell for example, a virus cell, a bacterial cell, or a eukaryotic cell (e.g., an insect cell, a plant cell, or an animal cell, such as a mammalian cell) can be used with no limitation. For example, the promoter may be at least one selected from the group consisting of promoters of prokaryotic cells or mammalian viruses, such as human CMV (human cytomegalo virus; hCMV) promoter, SV40 promoter, adenovirus promoter (major late promoter), pL^{λ} promoter, trp promoter, lac promoter, tac promoter, T7 promoter, vaccinia virus 7.5K promoter, HSV tk promoter, and the like, and promoters of animal cells, such as metallothionein promoter, beta-actin promoter, and the like.

In an embodiment, the promoter may be a human CMV (hCMV) promoter or a part thereof. The human CMV promoter may be i) a polynucleotide comprising or consisting essentially of a human CMV immediate-early enhancer/promoter (human CMV IE enhancer/promoter), ii) a polynucleotide fragment of a human CMV IE enhancer/promoter comprising or consisting essentially of consecutive nucleotide residues of at least about 100 bp, at least about 200 bp, at least about 300 bp, at least about 400 bp, or at least about 500 bp, for example, a consecutive nucleotide residues of about 100 to about 1000 bp, about 200 to about 900 bp, about 300 to about 800 bp, about 400 to about 750 bp, or about 500 to about 750 bp, within human CMV IE enhancer/promoter, or iii) a polynucleotide variant of human CMV IE enhancer/promoter (maintaining the function as a promoter) having a sequence identity of at least about 80%, at least about 85%, at least about 90%, at least about 95%, at least about 98%, or at least about 99% with the sequence of the polynucleotide fragment i) or ii).

For example, the polynucleotide fragment i) comprising human CMV IE enhancer/promoter may comprise or consist essentially of the nucleotide sequence of GenBank Accession No. X03922.1 (1848 bp; SEQ ID NO: 116):

| GenBank Accession No. X03922.1 (1848 bp; SEQ ID NO: 116) | |
|---|---|
| CTGCAGTGAA TAATAAAATG TGTGTTTGTC CGAAATACGC GTTTGAGATT | 50 |
| TCTGTCCCGA CTAAATTCAT GTCGCGCGAT AGTGGTGTTT ATCGCCGATA | 100 |
| GAGATGGCGA TATTGGAAAA ATCGATATTT GAAAATATGG CATATTGAAA | 150 |
| ATGTCGCCGA TGTGAGTTTC TGTGTAACTG ATATCGCCAT TTTTCCAAAA | 200 |
| GTTGATTTTT GGGCATACGC GATATCTGGC GATACGCTTA TATCGTTTAC | 250 |
| GGGGGATGGC GATAGACGCC TTTGGTGACT TGGGCGATTC TGTGTGTCGC | 300 |
| AAATATCGCA GTTTCGATAT AGGTGACAGA CGATATGAGG CTATATCGCC | 350 |
| GATAGAGGCG ACATCAAGCT GGCACATGGC CAATGCATAT CGATCTATAC | 400 |
| ATTGAATCAA TATTGGCCAT TAGCCATATT ATTCATTGGT TATATAGCAT | 450 |
| AAATCAATAT TGGCTATTGG CCATTGCATA CGTTGTATCC ATATCATAAT | 500 |
| ATGTACATTT ATATTGGCTC ATGTCCAACA TTACCGCCAT GTTGACATTG | 550 |
| ATTATTGACT AGTTATTAAT AGTAATCAAT TACGGGGTCA TTAGTTCATA | 600 |
| GCCCATATAT GGAGTTCCGC GTTACATAAC TTACGGTAAA TGGCCCGCCT | 650 |
| GGCTGACCGC CCAACGACCC CCGCCCATTG ACGTCAATAA TGACGTATGT | 700 |
| TCCCATAGTA ACGCCAATAG GGACTTTCCA TTGACGTCAA TGGGTGGAGT | 750 |
| ATTTACGGTA AACTGCCCAC TTGGCAGTAC ATCAAGTGTA TCATATGCCA | 800 |
| AGTACGCCCC CTATTGACGT CAATGACGGT AAATGGCCCG CCTGGCATTA | 850 |
| TGCCCAGTAC ATGACCTTAT GGGACTTTCC TACTTGGCAG TACATCTACG | 900 |
| TATTAGTCAT CGCTATTACC ATGGTGATGC GGTTTTGGCA GTACATCAAT | 950 |
| GGGCGTGGAT AGCGGTTTGA CTCACGGGGA TTTCCAAGTC TCCACCCCAT | 1000 |
| TGACGTCAAT GGGAGTTTGT TTTGGCACCA AAATCAACGG GACTTTCCAA | 1050 |
| AATGTCGTAA CAACTCCGCC CCATTGACGC AAATGGGCGG TAGGCGTGTA | 1100 |
| CGGTGGGAGG TCTATATAAG CAGAGCTCGT TTAGTGAACC GTCAGATCGC | 1150 |
| CTGGAGACGC CATCCACGCT GTTTTGACCT CCATAGAAGA CACCGGGACC | 1200 |
| GATCCAGCCT CCGCGGCCGG GAACGGTGCA TTGGAACGCG GATTCCCCGT | 1250 |
| GCCAAGAGTG ACGTAAGTAC CGCCTATAGA GTCTATAGGC CCACCCCCTT | 1300 |
| GGCTTCTTAT GCATGCTATA CTGTTTTTGG CTTGGGGTCT ATACACCCCC | 1350 |
| GCTTCCTCAT GTTATAGGTG ATGGTATAGC TTAGCCTATA GGTGTGGGTT | 1400 |
| ATTGACCATT ATTGACCACT CCCCTATTGG TGACGATACT TTCCATTACT | 1450 |
| AATCCATAAC ATGGCTCTTT GCACAACTCT CTTTATTGGC TATATGCCAA | 1500 |
| TACACTGTCC TTCAGAGACT GACACGGACT CTGTATTTTT ACAGGATGGG | 1550 |
| GTCTCATTTA TTATTTACAA ATTCACATAT ACAACACCAC CGTCCCCAGT | 1600 |
| GCCCGCAGTT TTTATTAAAC ATAACGTGGG ATCTCCAGCG AATCTCGGGT | 1650 |
| ACGTGTTCCG GACATGGGGC TCTTCTCCGG TAGCGGCGGA GCTTCTACAT | 1700 |
| CCAGCCCTGC TCCCATCCTC CCACTCATGG TCCTCGGCAG CTCCTTGCTC | 1750 |
| CTAACAGTGG AGGCCAGACT TAGGCACAGC ACGATGCCCA CCACCACCAG | 1800 |
| TGTGCCCACA AGGCCGTGGC GGTAGGGTAT GTGTCTGAAA ATGAGCTC | 1848 |

The polynucleotide fragment ii) may be a polynucleotide fragment comprising or consisting essentially of consecutive nucleotide residues within a region from position 400 to position 1250 of SEQ ID NO: 116 (X03922.1), in length of at least about 100 bp, at least about 200 bp, at least about 300 bp, at least about 400 bp, or at least about 500 bp, for example about 100 to about 850 bp, about 200 to about 800 bp, about 300 to about 750 bp, about 400 to about 750 bp, about 400 to about 750 bp, or about 500 to about 750 bp. For example, the polynucleotide fragment ii) may comprise or consist essentially of consecutive nucleotide residues of least about 100 bp, at least about 200 bp, at least about 300 bp, at least about 400 bp, or at least about 500 bp, for example, about 100 to about 850 bp, about 200 to about 800 bp, about 300 to about 750 bp, about 400 to about 750 bp, about 450 to about 750 bp, or about 500 to about 750 bp, in 3′-terminal direction starting from one selected from the nucleotide resides from position 400 to position 650 of SEQ ID NO: 116. For example, the polynucleotide fragment ii) may comprise or consist essentially of consecutive nucleotide residues of at least about 100 bp, at least about 200 bp, at least about 300 bp, at least about 400 bp, or at least about 500 bp, for example, about 100 to about 850 bp, about 200 to about 800 bp, about 300 to about 750 bp, about 400 to about 750 bp, about 450 to about 750 bp, or about 500 to about 750 bp, in 3′-terminal direction starting from one selected from the nucleotide resides from position 400 to position 410, from position 530 to position 550, or from position 615 to position 625, of SEQ ID NO: 116. For example, the polynucleotide fragment ii) may comprise or consist essentially of the nucleotide sequence of SEQ ID NO: 119, 120 or 121, or consecutive nucleotide residues of at least about 200 bp, at least about 300 bp or at least about 400 bp, of SEQ ID NO: 119, 120 or 121.

The polynucleotide variant iii) may be a variant of the polynucleotide fragment i) or ii), having a sequence identity of at least about 80%, at least about 85%, at least about 90%, at least about 95%, at least about 98%, or at least about 99%, with the sequence of the polynucleotide fragment i) or ii), and maintaining the function as a promoter. For example, the polynucleotide variant iii) may be:

iii-1) a polynucleotide variant comprising at least one mutation selected from the group consisting of substitution and deletion of at least one nucleotide in the polynucleotide fragment i) or ii), and insertion of a nucleotide into at least one position of the polynucleotide fragment i) or ii); or

iii-2) a polynucleotide variant comprising the nucleotide sequence of the polynucleotide fragment i) or ii) or the polynucleotide variant iii-1), in total length of about 200 to about 2000 bp, about 200 to about 1000 bp, about 200 to about 800 bp, about 200 to about 750 bp, about 300 to about 2000 bp, about 300 to about 1500 bp, about 300 to about 1000 bp, about 300 to about 800 bp, about 300 to about 750 bp, about 400 to about 2000 bp, about 400 to about 1500 bp, about 400 to about 1000 bp, about 400 to about 800 bp, about 400 to about 750 bp, about 500 to about 2000 bp, about 500 to about 1500 bp, about 500 to about 1000 bp, about 500 to about 800 bp, or about 500 to about 750 bp, wherein the nucleotides added to the polynucleotide fragment i) or ii) or the polynucleotide fragment iii-1) may be independently selected from the group consisting of A, T, G and C.

In an embodiment, the substitution in the polynucleotide variant iii-1) may be a substitution of at least one selected from the nucleotide residues of the polynucleotide fragment i) or ii) corresponding to positions 490 (C), 529 (C), 532 (T), 545 (A), 504 (A), 651 (G), 804 (A), 870 (T), 946 (T), 1061 (C), 1065 (T), and 1073 (A) of SEQ ID NO: 116, with a nucleotide(s) different from the original nucleotide(s). For example, polynucleotide variant iii-1) may comprise a substitution of at least one nucleotide in the polynucleotide fragment i) or ii), wherein the substitution may be at least one selected from the group consisting of:

a substitution of nucleotide C of the polynucleotide fragment i) or ii) corresponding to the position 490 of SEQ ID NO: 116 with T (C490T),

a substitution of nucleotide T of the polynucleotide fragment i) or ii) corresponding to the position 532 of SEQ ID NO: 116 with G (T532G),

a substitution of nucleotide A of the polynucleotide fragment i) or ii) corresponding to the position 545 of SEQ ID NO: 116 with G (A545G),

a substitution of nucleotide G of the polynucleotide fragment i) or ii) corresponding to the position 651 of SEQ ID NO: 116 with C (G651C),

a substitution of nucleotide A of the polynucleotide fragment i) or ii) corresponding to the position 804 of SEQ ID NO: 116 with C (A804C),

a substitution of nucleotide T of the polynucleotide fragment i) or ii) corresponding to the position 870 of SEQ ID NO: 116 with C (T870C),

a substitution of nucleotide T of the polynucleotide fragment i) or ii) corresponding to the position 946 of SEQ ID NO: 116 with C (T946C),

a substitution of nucleotide C of the polynucleotide fragment i) or ii) corresponding to the position 1061 of SEQ ID NO: 116 with T (C1061T),

a substitution of nucleotide T of the polynucleotide fragment i) or ii) corresponding to the position 1065 of SEQ ID NO: 116 with C (T1065C), and

a substitution of nucleotide A of the polynucleotide fragment i) or ii) corresponding to the position 1073 of SEQ ID NO: 116 with G (A1073G).

In an embodiment, the deletion may be a deletion of a nucleotide (C) of the polynucleotide fragment i) or ii) corresponding to the position 653 of SEQ ID NO: 116 (X03922.1). The insertion may be a insertion of a nucleotide (A, T, G, or C, for example G) between the nucleotides of the polynucleotide fragment i) or ii) corresponding to the positions 805 and 806 of SEQ ID NO: 116 (X03922.1). For example, the polynucleotide variant iii-1) may comprise or consist essentially of the nucleotide sequence of SEQ ID NO: 118, 122, or 124, or consecutive nucleotide residues of at least about 200 bp, at least about 300 bp, or at least about 400 bp, within the nucleotide sequence of SEQ ID NO: 118, 122, or 124.

The polynucleotide variant iii-2) may further comprise nucleotides of about 1 to about 100 bp or about 5 to about 60 bp, for example, about 5 to about 20 bp or about 45 to about 60 bp, at 5′-end, 3′-end, or both ends, for example 3′-end, of the polynucleotide fragment i) or ii) or the polynucleotide variant iii-1), wherein each of the further comprised nucleotides may be independently selected from A, T, G, and C. For example, the further comprised nucleotides may comprise or consist essentially of the nucleotide sequence of SEQ ID NO: 125 (ACTAGAAGCTTTATTGCGGTAGTTTATCACAGTTAAATTGCTAACGCAGTCAG) or SEQ ID NO: 126 (GACTCTA), but not be limited thereto. In an embodiment, the polynucleotide variant iii-2) may comprise or consist essentially of the nucleotide sequence of SEQ ID NO: 117 or 123, or consecutive nucleotide residues of at least about 200 bp, at least about 300 bp, or at least about 400 bp, within the nucleotide sequence of SEQ ID NO: 117 or 123.

The polynucleotide fragments and the polynucleotide variants are exemplified in Table 1:

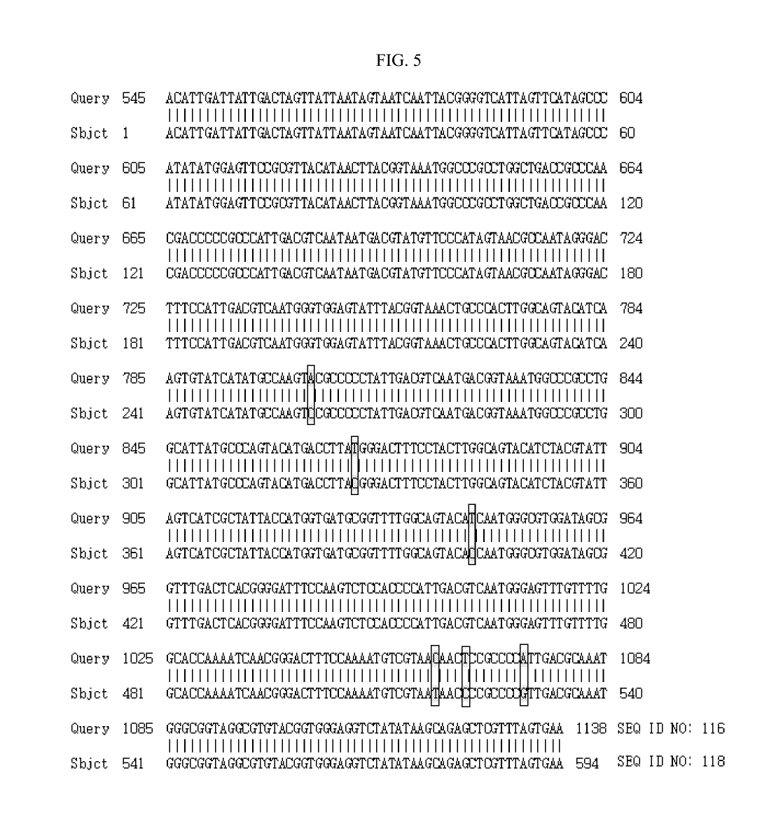

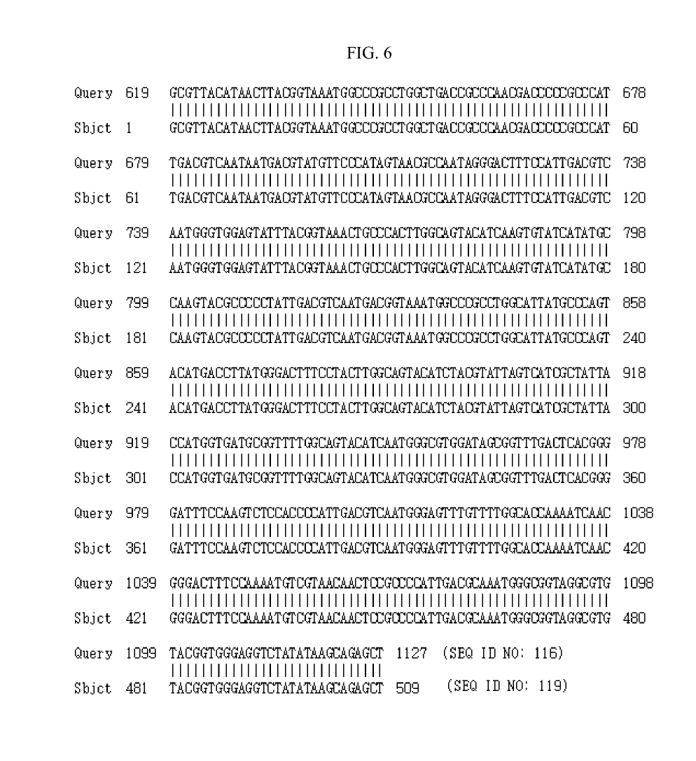

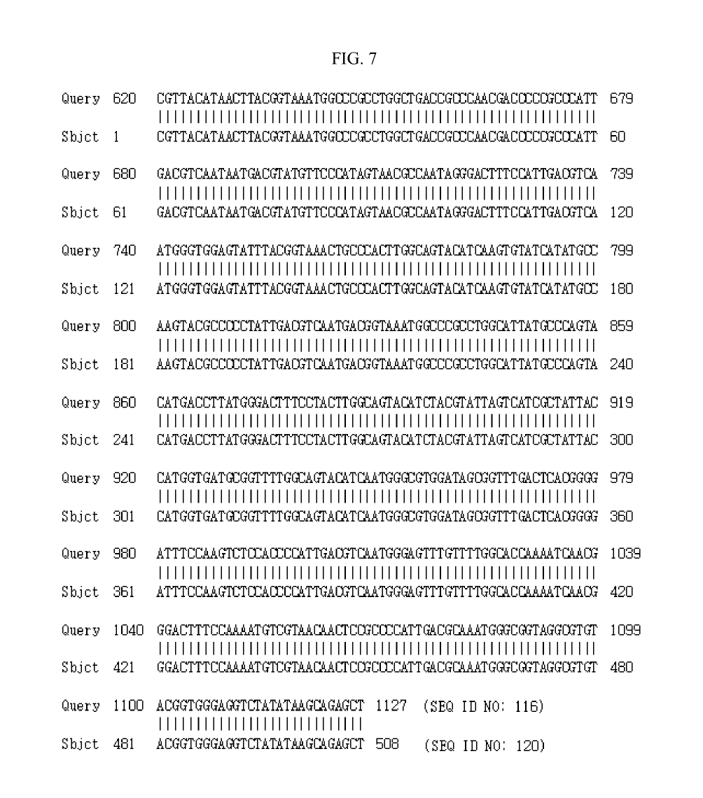

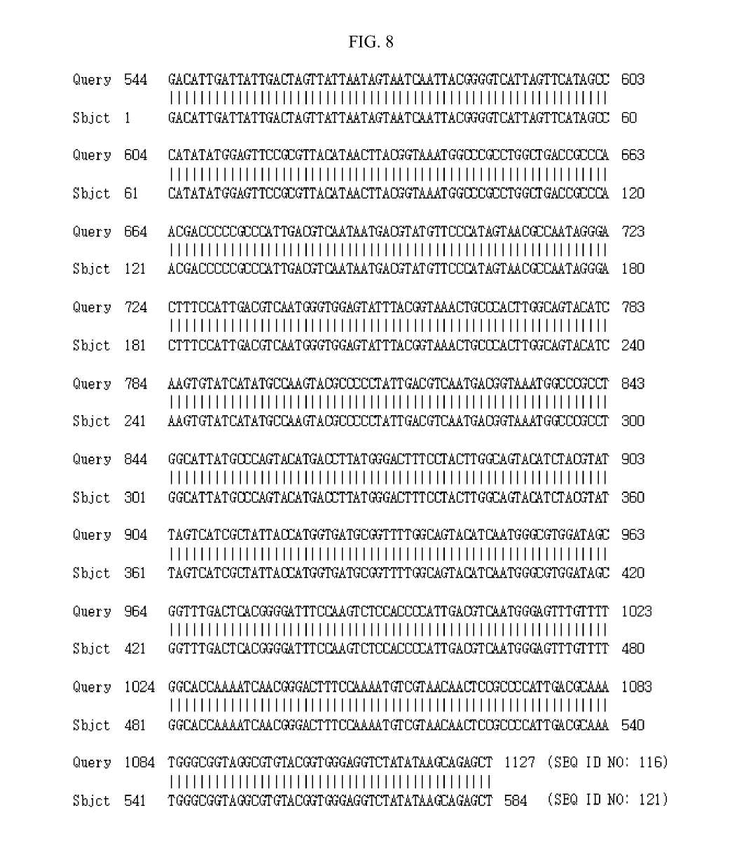

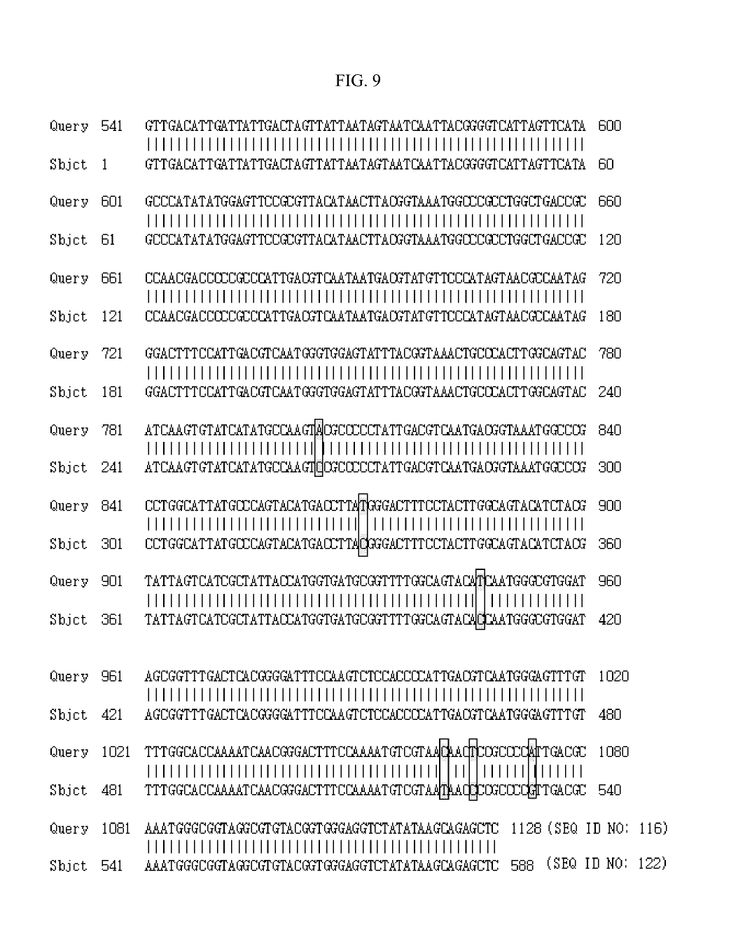

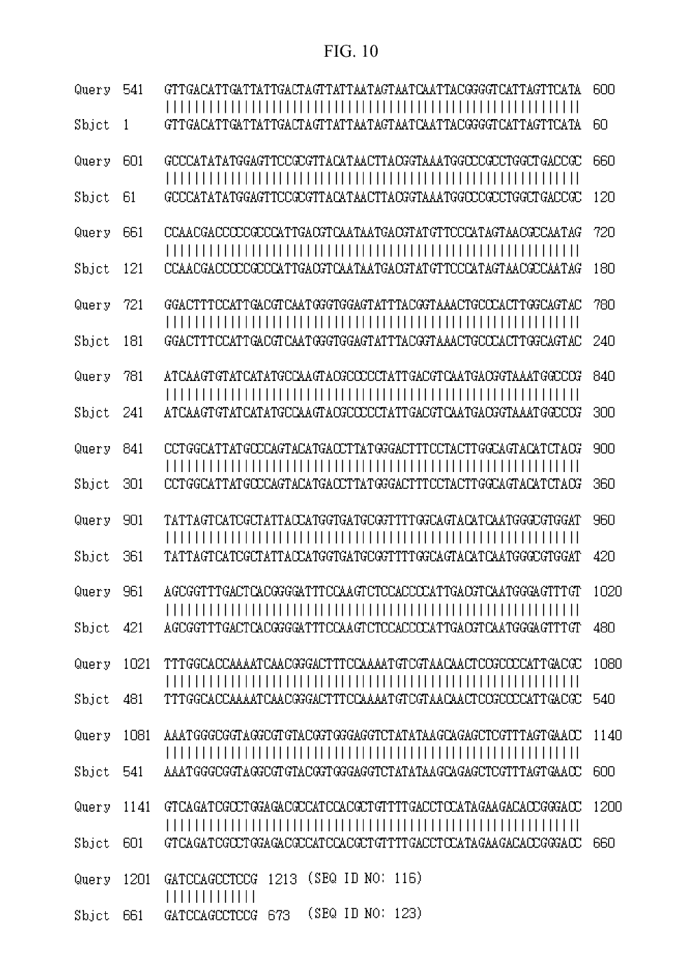

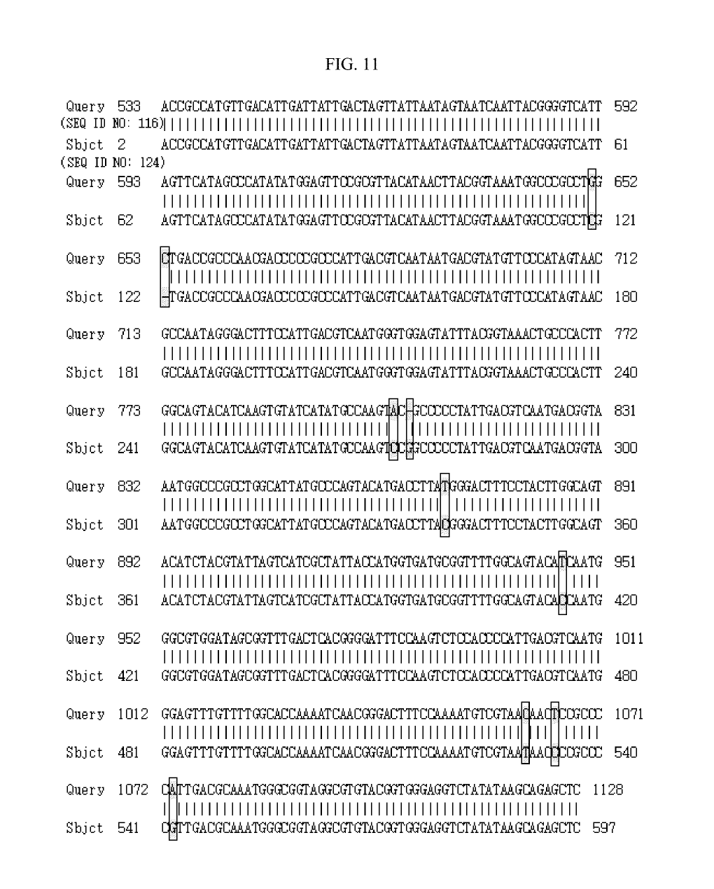

TABLE 1

| SEQ ID | (1) Position | (2) Sequence | | Nucleotide Sequence (the inserted |
|---|---|---|---|---|
| NO: | of X03922.1 | Identity | (3) Mutation | sequence into X03922.1 is underlined) |
| 117 | 407-1148 | 99% | C490T, | TCAATATTGGCCATTAGCCATATTATTCATTGG |
| (see FIG. | (742 bp) | (732/742) | C529T, | TTATATAGCATAAATCAATATTGGCTATTGGCC |
| 4) | | | T532G, | ATTGCATACGTTGTATCTATATCATAATATGTA |
| | | | A545G, | CATTTATATTGGCTCATGTCCAATATGACCGCC |
| | | | A804C, | ATGTTGGCATTGATTATTGACTAGTTATTAATA |
| | | | T870C, | GTAATCAATTACGGGGTCATTAGTTCATAGCCC |
| | | | T946C, | ATATATGGAGTTCCGCGTTACATAACTTACGGT |
| | | | C1061T, | AAATGGCCCGCCTGGCTGACCGCCCAACGACC |
| | | | T1065C, | CCCGCCCATTGACGTCAATAATGACGTATGTTC |
| | | | A1073G | CCATAGTAACGCCAATAGGGACTTTCCATTGAC |
| | | | & | GTCAATGGGTGGAGTATTTACGGTAAACTGCCC |
| | | | Addition of | ACTTGGCAGTACATCAAGTGTATCATATGCCAA |
| | | | ‘ACTAGAA | GTCCGCCCCCTATTGACGTCAATGACGGTAAAT |
| | | | GCTTTATT | GGCCCGCCTGGCATTATGCCCAGTACATGACCT |
| | | | GCGGTAG | TACGGGACTTTCCTACTTGGCAGTACATCTACG |
| | | | TTTATCAC | TATTAGTCATCGCTATTACCATGGTGATGCGGT |
| | | | AGTTAAA | TTTGGCAGTACACCAATGGGCGTGGATAGCGG |
| | | | TTGCTAA | TTTGACTCACGGGGATTTCCAAGTCTCCACCCC |
| | | | CGCAGTC | ATTGACGTCAATGGGAGTTTGTTTTGGCACCAA |
| | | | AG’(SEQ ID | AATCAACGGGACTTTCCAAAATGTCGTAATAA |
| | | | NO: 125) | CCCCGCCCCGTTGACGCAAATGGGCGGTAGGC |
| | | | at 3′- | GTGTACGGTGGGAGGTCTATATAAGCAGAGCT |
| | | | terminus | CGTTTAGTGAACCGTCAGATCACTAGAAGCTTT |
| | | | | ATTGCGGTAGTTTATCACAGTTAAATTGCTAAC |
| | | | | GCAGTCAG(795 bp) |
| 118 (see | 545-1138 | 99% | A804C, | ACATTGATTATTGACTAGTTATTAATAGTAATC |
| FIG. 5) | (594 bp) | (588/594) | T870C, | AATTACGGGGTCATTAGTTCATAGCCCATATAT |
| | | | T946C, | GGAGTTCCGCGTTACATAACTTACGGTAAATGG |
| | | | C1061T, | CCCGCCTGGCTGACCGCCCAACGACCCCCGCCC |
| | | | T1065C, | ATTGACGTCAATAATGACGTATGTTCCCATAGT |
| | | | A1073G | AACGCCAATAGGGACTTTCCATTGACGTCAATG |
| | | | | GGTGGAGTATTTACGGTAAACTGCCCACTTGGC |
| | | | | AGTACATCAAGTGTATCATATGCCAAGTCCGCC |
| | | | | CCCTATTGACGTCAATGACGGTAAATGGCCCGC |
| | | | | CTGGCATTATGCCCAGTACATGACCTTACGGGA |
| | | | | CTTTCCTACTTGGCAGTACATCTACGTATTAGT |
| | | | | CATCGCTATTACCATGGTGATGCGGTTTTGGCA |
| | | | | GTACACCAATGGGCGTGGATAGCGGTTTGACT |
| | | | | CACGGGGATTTCCAAGTCTCCACCCCATTGACG |
| | | | | TCAATGGGAGTTTGTTTTGGCACCAAAATCAAC |
| | | | | GGGACTTTCCAAAATGTCGTAATAACCCCGCCC |
| | | | | CGTTGACGCAAATGGGCGGTAGGCGTGTACGG |
| | | | | TGGGAGGTCTATATAAGCAGAGCTCGTTTAGTG |
| | | | | AA(594 bp) |
| 119 (see | 619-1127 | 100% | none | GCGTTACATAACTTACGGTAAATGGCCCGCCTG |
| FIG. 6) | (509 bp) | | | GCTGACCGCCCAACGACCCCCGCCCATTGACGT |
| | | | | CAATAATGACGTATGTTCCCATAGTAACGCCAA |
| | | | | TAGGGACTTTCCATTGACGTCAATGGGTGGAGT |
| | | | | ATTTACGGTAAACTGCCCACTTGGCAGTACATC |
| | | | | AAGTGTATCATATGCCAAGTACGCCCCCTATTG |
| | | | | ACGTCAATGACGGTAAATGGCCCGCCTGGCAT |
| | | | | TATGCCCAGTACATGACCTTATGGGACTTTCCT |
| | | | | ACTTGGCAGTACATCTACGTATTAGTCATCGCT |
| | | | | ATTACCATGGTGATGCGGTTTTGGCAGTACATC |
| | | | | AATGGGCGTGGATAGCGGTTTGACTCACGGGG |
| | | | | ATTTCCAAGTCTCCACCCCATTGACGTCAATGG |
| | | | | GAGTTTGTTTTGGCACCAAAATCAACGGGACTT |
| | | | | TCCAAAATGTCGTAACAACTCCGCCCCATTGAC |
| | | | | GCAAATGGGCGGTAGGCGTGTACGGTGGGAGG |
| | | | | TCTATATAAGCAGAGCT(509 bp) |
| 120 (see | 620-1127 | 100% | none | CGTTACATAACTTACGGTAAATGGCCCGCCTGG |
| FIG. 7) | (508 bp) | | | CTGACCGCCCAACGACCCCCGCCCATTGACGTC |
| | | | | AATAATGACGTATGTTCCCATAGTAACGCCAAT |
| | | | | AGGGACTTTCCATTGACGTCAATGGGTGGAGT |
| | | | | ATTTACGGTAAACTGCCCACTTGGCAGTACATC |
| | | | | AAGTGTATCATATGCCAAGTACGCCCCCTATTG |
| | | | | ACGTCAATGACGGTAAATGGCCCGCCTGGCAT |
| | | | | TATGCCCAGTACATGACCTTATGGGACTTTCCT |
| | | | | ACTTGGCAGTACATCTACGTATTAGTCATCGCT |
| | | | | ATTACCATGGTGATGCGGTTTTGGCAGTACATC |
| | | | | AATGGGCGTGGATAGCGGTTTGACTCACGGGG |
| | | | | ATTTCCAAGTCTCCACCCCATTGACGTCAATGG |
| | | | | GAGTTTGTTTTGGCACCAAAATCAACGGGACTT |
| | | | | TCCAAAATGTCGTAACAACTCCGCCCCATTGAC |
| | | | | GCAAATGGGCGGTAGGCGTGTACGGTGGGAGG |
| | | | | TCTATATAAGCAGAGCT(508 bp) |
| 121 (see | 544-1127 | 100% | none | GACATTGATTATTGACTAGTTATTAATAGTAAT |
| FIG. 8) | (584 bp) | | | CAATTACGGGGTCATTAGTTCATAGCCCATATA |
| | | | | TGGAGTTCCGCGTTACATAACTTACGGTAAATG |
| | | | | GCCCGCCTGGCTGACCGCCCAACGACCCCCGC |
| | | | | CCATTGACGTCAATAATGACGTATGTTCCCATA |
| | | | | GTAACGCCAATAGGGACTTTCCATTGACGTCAA |
| | | | | TGGGTGGAGTATTTACGGTAAACTGCCCACTTG |
| | | | | GCAGTACATCAAGTGTATCATATGCCAAGTAC |
| | | | | GCCCCCTATTGACGTCAATGACGGTAAATGGCC |
| | | | | CGCCTGGCATTATGCCCAGTACATGACCTTATG |
| | | | | GGACTTTCCTACTTGGCAGTACATCTACGTATT |
| | | | | AGTCATCGCTATTACCATGGTGATGCGGTTTTG |
| | | | | GCAGTACATCAATGGGCGTGGATAGCGGTTTG |
| | | | | ACTCACGGGGATTTCCAAGTCTCCACCCCATTG |
| | | | | ACGTCAATGGGAGTTTGTTTTGGCACCAAAATC |
| | | | | AACGGGACTTTCCAAAATGTCGTAACAACTCC |
| | | | | GCCCCATTGACGCAAATGGGCGGTAGGCGTGT |
| | | | | ACGGTGGGAGGTCTATATAAGCAGAGCT(584 bp) |
| 122 (see | 541-1128 | 99% | A804C, | GTTGACATTGATTATTGACTAGTTATTAATAGT |
| FIG. 9) | (588 bp) | (582/588) | T870C, | AATCAATTACGGGGTCATTAGTTCATAGCCCAT |
| | | | T946C, | ATATGGAGTTCCGCGTTACATAACTTACGGTAA |
| | | | C1061T, | ATGGCCCGCCTGGCTGACCGCCCAACGACCCC |
| | | | T1065C, | CGCCCATTGACGTCAATAATGACGTATGTTCCC |
| | | | A1073G | ATAGTAACGCCAATAGGGACTTTCCATTGACGT |
| | | | | CAATGGGTGGAGTATTTACGGTAAACTGCCCA |
| | | | | CTTGGCAGTACATCAAGTGTATCATATGCCAAG |
| | | | | TCCGCCCCCTATTGACGTCAATGACGGTAAATG |
| | | | | GCCCGCCTGGCATTATGCCCAGTACATGACCTT |
| | | | | ACGGGACTTTCCTACTTGGCAGTACATCTACGT |
| | | | | ATTAGTCATCGCTATTACCATGGTGATGCGGTT |
| | | | | TTGGCAGTACACCAATGGGCGTGGATAGCGGT |
| | | | | TTGACTCACGGGGATTTCCAAGTCTCCACCCCA |
| | | | | TTGACGTCAATGGGAGTTTGTTTTGGCACCAAA |
| | | | | ATCAACGGGACTTTCCAAAATGTCGTAATAACC |
| | | | | CCGCCCCGTTGACGCAAATGGGCGGTAGGCGT |
| | | | | GTACGGTGGGAGGTCTATATAAGCAGAGCTC |
| | | | | (588 bp) |
| 123 (see | 541-1213 | 100% | Addition of | GTTGACATTGATTATTGACTAGTTATTAATAGT |
| FIG. 10) | (673 bp) | | ‘GACTCTA’ | AATCAATTACGGGGTCATTAGTTCATAGCCCAT |
| | | | (SEQ ID | ATATGGAGTTCCGCGTTACATAACTTACGGTAA |
| | | | NO: 126) at | ATGGCCCGCCTGGCTGACCGCCCAACGACCCC |
| | | | 3′-end | CGCCCATTGACGTCAATAATGACGTATGTTCCC |
| | | | | ATAGTAACGCCAATAGGGACTTTCCATTGACGT |
| | | | | CAATGGGTGGAGTATTTACGGTAAACTGCCCA |
| | | | | CTTGGCAGTACATCAAGTGTATCATATGCCAAG |
| | | | | TACGCCCCCTATTGACGTCAATGACGGTAAATG |
| | | | | GCCCGCCTGGCATTATGCCCAGTACATGACCTT |
| | | | | ATGGGACTTTCCTACTTGGCAGTACATCTACGT |
| | | | | ATTAGTCATCGCTATTACCATGGTGATGCGGTT |
| | | | | TTGGCAGTACATCAATGGGCGTGGATAGCGGT |
| | | | | TTGACTCACGGGGATTTCCAAGTCTCCACCCCA |
| | | | | TTGACGTCAATGGGAGTTTGTTTTGGCACCAAA |
| | | | | ATCAACGGGACTTTCCAAAATGTCGTAACAACT |
| | | | | CCGCCCCATTGACGCAAATGGGCGGTAGGCGT |
| | | | | GTACGGTGGGAGGTCTATATAAGCAGAGCTCG |
| | | | | TTTAGTGAACCGTCAGATCGCCTGGAGACGCC |
| | | | | ATCCACGCTGTTTTGACCTCCATAGAAGACACC |
| | | | | GGGACCGATCCAGCCTCCGGACTCTA(680 bp) |
| 124 (see | 532-1128 | 98% | T532G, | GACCGCCATGTTGACATTGATTATTGACTAGTT |
| FIG. 11) | (597 bp) | (588/597) | G651C, | ATTAATAGTAATCAATTACGGGGTCATTAGTTC |
| | | 2 Gaps | C653 | ATAGCCCATATATGGAGTTCCGCGTTACATAAC |
| | | | deletion, | TTACGGTAAATGGCCCGCCTCGTGACCGCCCAA |
| | | | A804C, | CGACCCCCGCCCATTGACGTCAATAATGACGTA |
| | | | Insertion of | TGTTCCCATAGTAACGCCAATAGGGACTTTCCA |
| | | | “G” between | TTGACGTCAATGGGTGGAGTATTTACGGTAAAC |
| | | | positions | TGCCCACTTGGCAGTACATCAAGTGTATCATAT |
| | | | 805 and 806, | GCCAAGTCCGGCCCCCTATTGACGTCAATGACG |
| | | | T870C, | GTAAATGGCCCGCCTGGCATTATGCCCAGTACA |
| | | | T946C, | TGACCTTACGGGACTTTCCTACTTGGCAGTACA |
| | | | C1061T, | TCTACGTATTAGTCATCGCTATTACCATGGTGA |
| | | | T1065C, | TGCGGTTTTGGCAGTACACCAATGGGCGTGGAT |
| | | | A1073G | AGCGGTTTGACTCACGGGGATTTCCAAGTCTCC |
| | | | | ACCCCATTGACGTCAATGGGAGTTTGTTTTGGC |
| | | | | ACCAAAATCAACGGGACTTTCCAAAATGTCGT |
| | | | | AATAACCCCGCCCCGTTGACGCAAATGGGCGG |
| | | | | TAGGCGTGTACGGTGGGAGGTCTATATAAGCA |
| | | | | GAGCTC(597 bp) |

(1) a region in SEQ ID NO: 116 (X03922.1) which has the sequence identity with each of the polynucleotide fragment and the polynucleotide variant,
(2) the sequence identity between the nucleotide sequences of the region 1) and each of the polynucleotide fragment and the polynucleotide variant, and
(3) the mutated position of the polynucleotide variant which is indicated based on SEQ ID NO: 116 (X03922.1).

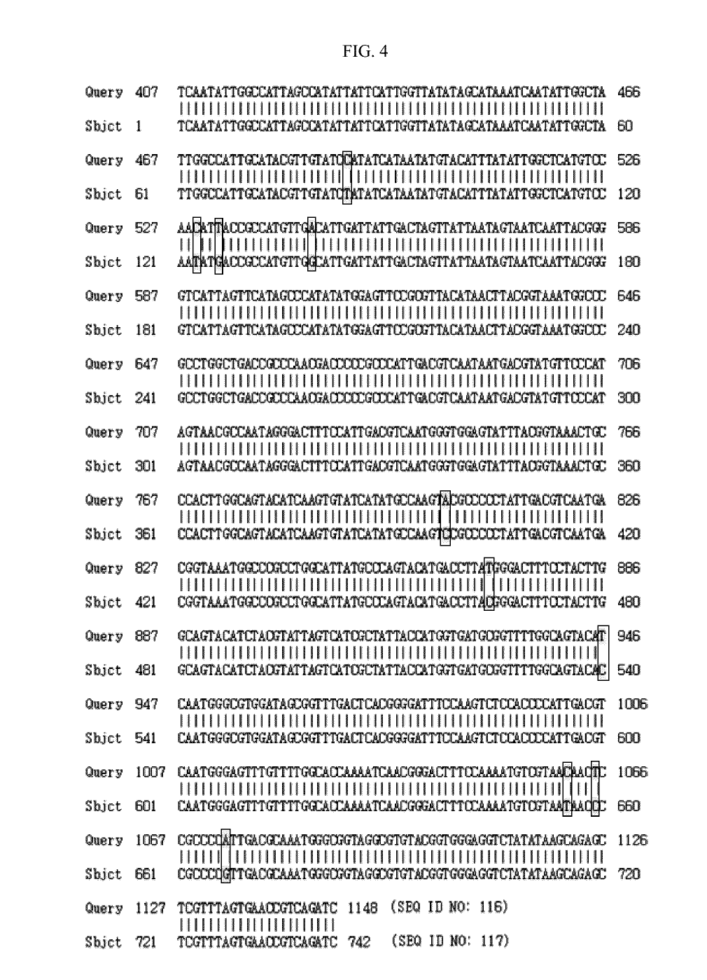

The position in each of the polynucleotide fragment and the polynucleotide variant indicated based on SEQ ID NO: 116 may be more clearly understood referring to FIGS. 4 to 11.

In an particular embodiment, the human CMV promoter may be 1) a polynucleotide fragment comprising SEQ ID NO: 124, 2) polynucleotide fragment comprising consecutive nucleotides of at least 200 bp within SEQ ID NO: 124, or 3) a polynucleotide fragment further comprising about 1 to about 100 nucleotides at 3′-end, 5′-end or both ends of the polynucleotide fragment 1) or 2).

The term “intron” may refer to a non-translated and intervening nucleotide sequence located between exons which are translated into a protein after transcription. A final mature RNA product is generated by removing the non-translated region, intron, from a transcribed mRNA precursor by RNA splicing.

The intron may be any intron isolated from a gene of an animal, for example, a mammal (e.g., human). The intron may be at least one selected from the group consisting of immunoglobulin introns (e.g., at least one selected from the group consisting of IGLV intron, IGKV intron, IGH intron, and the like), a chimeric intron, an intron A of human cytomegalovirus major immediate-early release protein gene, and the like. For example, the intron may be at least one selected from the group consisting of IGLV intron, IGKV intron, and IGH intron.

IGLV (immunoglobulin lambda variable) intron refers to an intron region of a gene encoding a variable region of lambda light chain of an immunoglobulin. For example, the IGLV intron may be a human IGLV intron (IGLV-1L1; e.g., an intron from position 71 to position 185 of Accession No. X59707; 115 bp; SEQ ID NO: 109); an intron fragment comprising or consisting essentially of consecutive nucleotide residues of about 10 to about 115 bp, about 30 to about 115 bp, about 50 to about 115 bp, about 80 to about 115 bp, about 100 to about 115 bp, about 10 to about 114 bp, about 30 to about 114 bp, about 50 to about 114 bp, about 80 to about 114 bp, or about 100 to about 114 bp within SEQ ID NO: 109; an intron variant of the intron of SEQ ID NO: 109 or an intron fragment thereof, having a sequence identity of at least about 80%, at least about 85%, at least about 90%, at least about 95%, at least about 98%, or at least about 99%, with the sequence of the intron or intron fragment; or an intron variant of the intron of SEQ ID NO: 109 or an intron fragment thereof, wherein nucleotide residues (each nucleotide is independently selected from A, T, G, and C) of about 1 to about 50 bp or about 10 to about 30 bp are added to 5′-end, 3′-end, or both ends of the intron or intron fragment.

IGKV (immunoglobulin kappa variable) intron refers to an intron region of a gene encoding a variable region of kappa light chain of an immunoglobulin. For example, the IGKV intron may be a human IGKV intron (e.g., an intron from position 269 to position 474 of Accession No. M27751.1 or X12688.1; 206 bp; SEQ ID NO: 110); an intron fragment comprising or consisting essentially of consecutive nucleotide residues of about 100 to about 206 bp, about 130 to about 206 bp, about 150 to about 206 bp, about 180 to about 206 bp, about 200 to about 206 bp, about 100 to about 205 bp, about 130 to about 205 bp, about 150 to about 205 bp, about 180 to about 205 bp, or about 200 to about 205 bp within SEQ ID NO: 110; an intron variant of the intron of SEQ ID NO: 110 or an intron fragment thereof, having a sequence identity of at least about 80%, at least about 85%, at least about 90%, at least about 95%, at least about 98%, or at least about 99%, with the sequence of the intron or intron fragment; or an intron variant of the intron of SEQ ID NO: 110 or an intron fragment thereof, wherein nucleotide residues (each nucleotide is independently selected from A, T, G, and C) of about 1 to about 50 bp or about 10 to about 30 bp are added to 5′-end, 3′-end, or both ends of the intron or intron fragment.

IGH (immunoglobulin heavy locus) intron refers to an intron region of a gene encoding a heavy chain of an immunoglobulin. The IGH intron may be obtained from any isotype of immunoglobulins, such as IgA, IgD, IgG, IgM, or IgE. For example, the IGH intron may be a human IGH intron (e.g., an intron from position 197 to position 278 of Accession No. M29811; 82 bp; SEQ ID NO: 111); an intron fragment comprising or consisting essentially of consecutive nucleotide residues of about 10 to about 82 bp, about 30 to about 82 bp, about 50 to about 82 bp about 10 to about 81 bp, about 30 to about 81 bp, or about 50 to about 81 bp, within SEQ ID NO: 111; an intron variant of the intron of SEQ ID NO: 111 or an intron fragment thereof, having a sequence identity of at least about 80%, at least about 85%, at least about 90%, at least about 95%, at least about 98%, or at least about 99%, with the sequence of the intron or intron fragment; or an intron variant of the intron of SEQ ID NO: 111 or an intron fragment thereof, wherein nucleotide residues (each nucleotide is independently selected from A, T, G, and C) of about 1 to about 50 bp or about 10 to about 30 bp are added to 5′-end, 3′-end, or both ends of the intron or intron fragment.

For example, the chimeric intron may be an intron comprising or consisting essentially of the nucleotide sequence of SEQ ID NO: 112 (133 bp); an intron fragment comprising or consisting essentially of consecutive nucleotide residues of about 10 to about 133 bp, about 30 to about 133 bp, about 50 to about 133 bp, about 80 to about 133 bp, about 100 to about 133 bp, about 10 to about 132 bp, about 30 to about 132 bp, about 50 to about 132 bp, about 80 to about 132 bp, or about 100 to about 132 bp, within SEQ ID NO: 112; an intron variant of the intron of SEQ ID NO: 112 or an intron fragment thereof, having a sequence identity of at least about 80%, at least about 85%, at least about 90%, at least about 95%, at least about 98%, or at least about 99%, with the sequence of the intron or intron fragment; or an intron variant of the intron of SEQ ID NO: 112 or an intron fragment thereof, wherein nucleotide residues (each nucleotide is independently selected from A, T, G, and C) of about 1 to about 50 bp or about 10 to about 30 bp are added to 5′-end, 3′-end, or both ends of the intron or intron fragment.

For example, intron A may be human intron A (e.g., Chapman et al., Nucleic Acids Res. 1991 Jul. 25; 19(14): 3979-3986; SEQ ID NO: 113 (963 bp)); an intron fragment comprising or consisting essentially of consecutive nucleotide residues of about 100 to about 963 bp, about 300 to about 963 bp, about 500 to about 963 bp, about 800 to about 963 bp, about 900 to about 963 bp, about 100 to about 962 bp, about 300 to about 962 bp, about 500 to about 962 bp, about 800 to about 962 bp, or about 900 to about 962 bp, within SEQ ID NO: 113; an intron variant of the intron of SEQ ID NO: 113 or an intron fragment thereof, having a sequence identity of at least about 80%, at least about 85%, at least about 90%, at least about 95%, at least about 98%, or at least about 99%, with the sequence of the intron or intron fragment; or an intron variant of the intron of SEQ ID NO: 113 or an intron fragment thereof, wherein nucleotide residues (each nucleotide is independently selected from A, T, G, and C) of about 1 to about 50 bp or about 10 to about 30 bp are added to 5′-end, 3′-end, or both ends of the intron or intron fragment.

In the fusion polynucleotide or fusion promoter, the intron may be linked to 5′-terminus, 3′-terminus, or both ends (in case two or more introns, which is the same with or different from each other, are linked) of the promoter (i.e., a polynucleotide fragment or a polynucleotide variant as described above). For example, the intron may be linked to 3′-terminus of the promoter (i.e., the promoter is located at 5′-terminal part and the intron is located at 3′-terminal part in the fusion protein). In the fusion polynucleotide or fusion promoter, the promoter and the intron may be linked to each other directly or via a proper linker. The linker may be any oligonucleotide, e.g., in length of 2-30 bp, 2-20 bp, or 2-10 bp, but not be limited thereto.

Another embodiment provides a method of preparing a fusion promoter, comprising linking an intron to 5′-terminus or 3′-terminus (e.g., 3′-terminus) of a promoter, or liking at least two introns, which are the same as or different from each other, to both ends of the promoter. The fusion promoter may be capable of operating in an animal cell, for example, a mammalian cell. The details of the promoter and intron are as described above.

The fusion polynucleotide or fusion promoter may function to effectively initiate transcription of a gene which is operatively linked thereto. The fusion polynucleotide or fusion promoter may be capable of effectively initiating transcription in any host cell, for example, a viral cell, a bacterial cell, or a eukaryotic cell, such as an insect cell, a plant cell, or an animal cell (e.g., a mammalian cell). For example, the fusion polynucleotide or fusion promoter may be capable of effectively initiating transcription in an animal cell, such as, a mammalian cell. The mammalian cell may be at least one selected from the group consisting of a mouse cell (e.g., COP, L, C127, Sp2/0, NS-0, NS-1, At20, NIH3T3, etc.), a rat cell (e.g., PC12, PC12h, GH3, MtT, etc.), a hamster cell (e.g., BHK, CHO, GS (glutamine synthetase) gene deficient CHO, DHFR (dihydrofolate reductase) gene deficient CHO, etc.), a monkey cell (e.g., COS1, COS3, COS7, CV1, Vero, etc.), a human cell (e.g., Hela, HEK-293, PER C6 cell derived from retinal tissue, a cell derived from diploid fibroblast, myeloma cell, HepG2, etc.), and the like.

Another embodiment provides a recombinant vector comprising the fusion polynucleotide. The recombinant vector may be useful as an expression vector of a polypeptide of interest capable of highly expressing the fusion polynucleotide in a proper host cell, when a gene (“a gene of interest”) encoding the polypeptide of interest is operatively linked to the fusion polynucleotide.

Another embodiment provides a recombinant vector comprising the fusion polynucleotide and a gene encoding a polypeptide of interest. In this case, the fusion polynucleotide may act as a promoter, and be operatively linked to the gene of interest.

The term “vector” refers to a means for expressing a target gene in a host cell. A vector may comprise elements necessary for expressing a gene of interest, such as a replication origin, a promoter, an operator, a terminator, and the like. In addition, a vector may further comprise at least one selected from the group consisting of an enzyme recognition site (e.g., a recognition (restriction) site of a restriction enzyme) for introducing a foreign gene into a genome of a host cell, a selection marker for confirming a successful introduction of the vector into a host cell, a ribosome binding site (RBS) for translation to a protein, an internal ribosome entry site (IRES), and the like. A vector may be genetically engineered so as to comprise the fusion polypeptide as a promoter. A vector may further comprise transcription control sequences (e.g., an enhancer) in addition to a promoter.

The vector may be exemplified by a plasmid vector, a cosmid vector, or a viral vector such as a bacteriophage vector, adenovirus vector, retrovirus vector, and an adeno-related virus vector. The recombinant vector may be constructed from, but not limited to, well-known plasmids (for example, pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, etc.), phages (for example, λgt4λB, λ-Charon, λAz1, M13, etc.) or viruses (for example, SV40, etc.) by manipulation.

In the recombinant vector, the gene of interest may be operatively linked to the fusion polynucleotide as a promoter. The term “operatively linked” is intended to pertain to a functional linkage between a nucleotide sequence (a gene) of interest and an expression regulatory element (for example, a promoter sequence) so that the expression of the nucleotide sequence of interest is controlled by the regulatory element. For instance, when the regulatory element such as a promoter is “operatively linked” to the nucleotide sequence (gene) of interest, it can control the transcription and/or translation of the nucleotide sequence (gene) of interest. In the recombinant vector, the fusion polynucleotide may be linked to 5′-end of a gene of interest, so that it can be operatively linked thereto.

The recombinant vector may be constructed by any method well-known in the art.

The recombinant vector may further comprise a transcription regulatory sequence in addition to a promoter. The transcription regulatory sequences may be at least one selected from the group consisting of a terminator, such as a polyadenylation sequence (pA; e.g., SEQ ID NO: 115); an origin of replication, such as an f1 origin of replication, an SV40 origin of replication, a pMB1 origin of replication, an adeno origin of replication, an AAV origin of replication, or a BBV origin of replication; and any combination thereof.

In addition, the recombinant vector may further comprise a selection marker. The selection marker may refer to a gene for confirming whether or not the recombinant vector is successfully introduced into a host cell or establishing a stable recombinant cell comprising the recombinant vector. The selection marker may be a drug-resistant gene (e.g., an antibiotic-resistant gene), a metabolism-related gene, a gene amplifying gene, or any combination thereof. The selection marker may not affect the expression efficiency of the vector, and thus it can be any drug-resistant gene (e.g., an antibiotic-resistant gene) and/or a metabolism-related gene, which is generally used for a recombinant vector. For example, the selection marker may be at least one selected from the group consisting of an ampicilin-resistant gene, a tetracyclin-resistant gene, a kanamycin-resistant gene, a chloroamphenicol-resistant gene, a streptomycin-resistant gene, a neomycin-resistant gene, a zeocin-resistant gene, a puromycin-resistant gene, a thymidine kinase (TK) gene, a dihydrofolate reductase (DHFR) gene, a glutamine synthetase (GS) gene, and the like, but not be limited thereto.

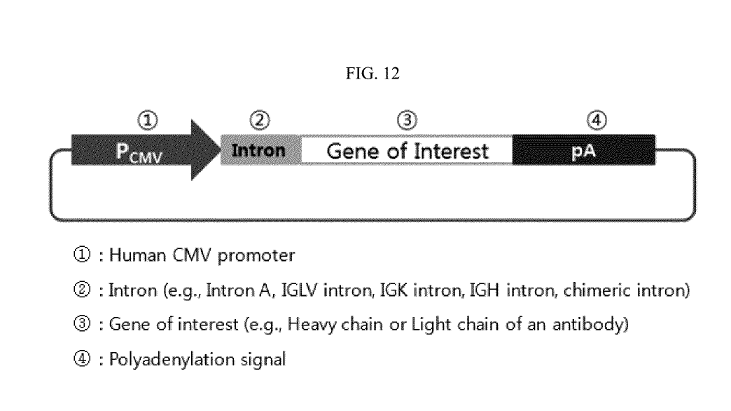

An example of the recombinant vector is illustrated in FIG. 12.

Another embodiment provides a recombinant cell comprising the recombinant vector. The recombinant cell may refer to a cell transfected with the recombinant vector, i.e., a cell generated by introducing the recombinant vector into a host cell. The recombinant cell may further comprise a polynucleotide (a gene of interest) encoding a polypeptide of interest. In this case, a gene of interest may be introduced into the host cell together with the fusion polynucleotide (fusion promoter) (e.g., comprising a human CMV promoter and intron), through one recombinant vector (i.e., comprising the fusion promoter and a gene of interest) or two separate recombinant vectors (i.e., both comprising the fusion promoter and a gene of interest).

The host cell for preparing the recombinant cell may be any animal cell (for example, any mammalian cell), wherein the fusion polynucleotide can act as a promoter (i.e., have a function to initiate transcription) and an expression of a gene of interest is allowed. For example, the host cell may be at least one mammalian cell selected from the group consisting of a mouse cell (e.g., COP, L, C127, Sp2/0, NS-0, NS-1, At20, NIH3T3, etc.), a rat cell (e.g., PC12, PC12h, GH3, MtT, etc.), a hamster cell (e.g., BHK, CHO, GS (glutamine synthetase) gene deficient CHO, DHFR (dihydrofolate reductase) gene deficient CHO, etc.), a monkey cell (e.g., COS1, COS3, COS7, CV1, Vero, etc.), a human cell (e.g., Hela, HEK-293, PER C6 cell derived from retinal tissue, a cell derived from diploid fibroblast, myeloma cell, HepG2, etc.), and the like. The host cell may be isolated (separated) from a living body.

Using a method well known in the art, the fusion polynucleotide or a recombinant vector carrying the fusion polynucleotide may be introduced (incorporated or transfected) into a host cell. This transfection may be carried out through CaCl2 or electroporation when the host cell is prokaryotic. For eukaryotic host cells, the genetic introduction may be performed using, but not limited to, microinjection, calcium phosphate precipitation, electroporation, liposome-mediated transfection, or particle bombardment.

To select a transfected host cell, advantage may be taken of the phenotype attributed to a selection marker according to a method known in the art. For example, when the selection marker is a gene resistant to a certain antibiotic as described above, the host cells may be grown in the presence of the antibiotic in a medium to select a transfected cell.

When the polypeptide of interest has an effect of preventing, treating, improving, and/or ameliorating a disease and/or a pathologic condition, an embodiment provides a pharmaceutical composition comprising at least one selected from the group consisting of a recombinant vector comprising the fusion polynucleotide and a gene encoding the polypeptide of interest, a recombinant cell comprising the recombinant vector, and a culture (in a cell-containing or cell-free form) of the recombinant cell.

In another embodiment, a use for the recombinant vector comprising the fusion polynucleotide and/or the recombinant cell comprising the recombinant vector, is to increase the production of a polypeptide of interest. In particular, provided is a composition for producing a polypeptide of interest, wherein the composition comprises a recombinant vector comprising the fusion polynucleotide and a gene encoding the polypeptide of interest which is operatively linked to the fusion polynucleotide, a recombinant cell comprising the recombinant vector, or a combination thereof.

Another embodiment provides a method of producing a polypeptide of interest using the recombinant vector or the recombinant cell.

For example, the method of producing a polypeptide of interest may comprise expressing a gene encoding a polypeptide of interest in the recombinant cell. The step of expressing a gene may be performed in vitro. The step of expressing a gene may comprise culturing the recombinant cell in a medium for the cell and under conditions allowing expression of the gene in the cell, wherein the medium and conditions may be clear to the relevant art. In addition, the method may further comprise harvesting (obtaining or separating) the polypeptide of interest from the expressing or culturing product, after the step of expressing or culturing. The step of harvesting the polypeptide of interest may be performed by separating the polypeptide from the recombinant cell, a lysate thereof, and/or a culture media (in case the polypeptide is secreted to a medium). The method of producing may further comprise an additional step, such as a step of purification and/or modification, so that the harvested polypeptide can have a desired quality and/or purity.

As used herein, the term “polypeptide” refers to a molecule covering a polymer of amino acids which are linked to one another through peptide bond(s). The polypeptide may a polypeptide in any length; for example, the polypeptide may be a protein (e.g., comprising about 50 or more amino acids) or a peptide (e.g., comprising about 2 to 49 amino acids).

The term “polypeptide of interest” may refer to a protein or a peptide having a desired activity (e.g., an activity of treating, preventing, and/or ameliorating a certain disease or symptom, and/or replacing a substance necessary in a living body) in a living body or cell. For example, the polypeptide of interest may be at least one selected from the group consisting of a protein or peptide having an enzymatic activity (e.g., a protease, a kinase, a phosphatase, etc.), a receptor protein or peptide, a transporter protein or peptide, a microbicidal and/or endotoxin-binding polypeptide, a structural protein or peptide, an immunoglobulin, a toxin, an antibiotic, a hormone, a growth factor, a vaccine, and the like. The polypeptide of interest or the gene of interest may be intrinsic (i.e., originally present in a host cell) or extrinsic (i.e., introduced from out of a host cell), and in case the polypeptide or gene is extrinsic, it may be introduced from the same species with or different species from the host cell.

In an embodiment, the polypeptide of interest may be at least one selected from the group consisting of a hormone, a cytokine, a tissue plasminogen activator, an immunoglobulin (e.g., an antibody or an antigen-binding fragment thereof or a variant thereof), and the like. The immunoglobulin (also refers to an antibody) may be any isotype (e.g., IgA, IgD, IgG, IgM or IgE), for example, IgG molecule (e.g., IgG1, IgG2, IgG3, or IgG4). The antigen-binding fragment refers to an antibody fragment possessing an antigen binding ability of the antibody, and may be comprise or consist essentially of at least about 20 amino acids, for example, at least about 100 amino acids. The antigen-binding fragment may be any fragment containing an antigen-binding region, and for example, it may be at least one selected from the group consisting of CDRs (complementarity determining regions), a Fab fragment, a Fab′ fragment, a F(ab)2 fragment, a F(ab′)2 fragment, a Fv fragment, a scFv fragment, a (scFv)2 fragment, a scFv-Fc fragment, a multibody containing various antigen-binding domains (e.g., a diabody, a triabody, a tetrabody, etc.), a single-domain antibody, an affibody, and the like. The variant of an antibody refers to a derivative of an antibody or an antibody fragment, which has an amino acid sequence modified from the amino acid sequence of an original antibody, with maintaining an antigen-binding ability of the original antibody. The antibody and/or antigen-binding fragment may be, but not limited to, animal antibodies (e.g., mouse-derived antibodies), chimeric antibodies (e.g., mouse-human chimeric antibodies), humanized antibodies, or human antibodies. The antibody or antigen-binding fragment may be isolated from a living body or non-naturally occurring (e.g., being synthetic or recombinant). The antibody may be monoclonal. When the polypeptide of interest is an antibody or antigen-binding fragment, the recombinant vector may comprise i) a gene encoding a heavy chain and/or a gene encoding a light chain, or gene encoding an antigen-binding fragment, and ii) a fusion promoter (fusion polynucleotide) which is operatively linked to the gene i). In this case, a gene encoding a heavy chain and a gene encoding a light chain may be carried together in one vector, or separately in different vectors. A recombinant vector containing both a gene encoding a heavy chain and a gene encoding a light chain, or at least two recombinant vectors, each of which contains each of a gene encoding a heavy chain and a gene encoding a light chain, can be introduced into a host cell. Alternatively, the polypeptide of interest may be at least one selected from the group consisting of insulin, human growth hormone (hGH), various growth factors, such as insulin-like growth factor, epidermal growth factor (EGF), vascular endothelial growth factor (VEGF), and the like, various receptors, tissue plasminogen activator (tPA), erythropoietin (EPO), cytokines (e.g., interleukin such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, and the like), interferon (IFN)-alpha, IFN-beta, IFN-gamma, IFN-omega or IFN-tau, tumor necrosis factors (TNF) such as TNF-alpha, TNF-beta or TNF-gamma, TRAIL, G-CSF, GM-CSF, M-CSF, MCP-1, and the like.

A gene encoding a polypeptide of interest (a gene of interest) may be intrinsic (i.e., originally present in a host cell) or extrinsic (i.e., introduced from out of a host cell), and in the case where the polypeptide or gene is extrinsic, it may be introduced from the same species as or a different species from the host cell. The details (e.g., a nucleotide sequence) of a gene of interest may be clearly defined by the described a polypeptide of interest.

In an embodiment, the polypeptide of interest may be an anti-c-Met antibody or an antigen-binding fragment thereof.

The anti-c-Met antibody or an antigen-binding fragment thereof may be any antibody which specifically recognizes c-Met as an antigen and/or specifically binds to c-Met, or an antigen-binding fragment thereof. For example, the anti-c-Met antibody or antigen-binding fragment thereof may be any antibody that acts on c-Met to induce intracellular internalization and degradation of c-Met. The anti-c-Met antibody may recognize any specific region of c-Met, e.g., a specific region in the SEMA domain, as an epitope.

“c-Met” or “c-Met protein” refers to a receptor tyrosine kinase (RTK) which binds hepatocyte growth factor (HGF). c-Met may be derived (obtained) from any species, particularly a mammal, for instance, primates such as human c-Met (e.g., GenBank Accession No. NP_{—}000236), monkey c-Met (e.g., Macaca mulatta, GenBank Accession No. NP_{—}001162100), or rodents such as mouse c-Met (e.g., GenBank Accession No. NP_{—}032617.2), rat c-Met (e.g., GenBank Accession No. NP_{—}113705.1), and the like. The c-Met protein may include a polypeptide encoded by the nucleotide sequence identified as GenBank Accession No. NM_{—}000245, a polypeptide having the amino acid sequence identified as GenBank Accession No. NP_{—}000236 or extracellular domains thereof. The receptor tyrosine kinase c-Met participates in various mechanisms, such as cancer incidence, metastasis, migration of cancer cells, invasion of cancer cells, angiogenesis, and the like.

c-Met, a receptor for hepatocyte growth factor (HGF), may be divided into three portions: extracellular, transmembrane, and intracellular. The extracellular portion is composed of an α-subunit and a β-subunit which are linked to each other through a disulfide bond, and includes a SEMA domain responsible for binding HGF, a PSI domain (plexin-semaphorins-integrin identity/homology domain) and an IPT domain (immunoglobulin-like fold shared by plexins and transcriptional factors domain). The SEMA domain of c-Met protein may have the amino acid sequence of SEQ ID NO: 79, and is an extracellular domain that functions to bind HGF. A specific region of the SEMA domain, that is, a region having the amino acid sequence of SEQ ID NO: 71, which corresponds to a range from amino acid residues 106 to 124 of the amino acid sequence of the SEMA domain (SEQ ID NO: 79), is a loop region between the second and the third propellers within the epitopes of the SEMA domain. This region acts as an epitope for the anti-c-Met antibody.

The term “epitope,” as used herein, refers to an antigenic determinant, a part of an antigen recognized by an antibody. In one embodiment, the epitope may be a region including 5 or more contiguous (consecutive on primary, secondary (two-dimensional), or tertiary (three-dimensional) structure) amino acid residues within the SEMA domain (SEQ ID NO: 79) of c-Met protein, for instance, 5 to 19 contiguous amino acid residues within the amino acid sequence of SEQ ID NO: 71. For example, the epitope may be a polypeptide having 5 to 19 contiguous amino acids selected from among partial combinations of the amino acid sequence of SEQ ID NO: 71, wherein the polypeptide includes at least the amino sequence of SEQ ID NO: 73 (EEPSQ) which serves as an essential element for the epitope. For example, the epitope may be a polypeptide including, consisting essentially of, or consisting of the amino acid sequence of SEQ ID NO: 71, SEQ ID NO: 72, or SEQ ID NO: 73.

The epitope having the amino acid sequence of SEQ ID NO: 72 corresponds to the outermost part of the loop between the second and third propellers within the SEMA domain of a c-Met protein. The epitope having the amino acid sequence of SEQ ID NO: 73 is a site to which the antibody or antigen-binding fragment according to one embodiment most specifically binds.

Thus, the c-Met inhibitor may specifically bind to an epitope which has 5 to 19 contiguous amino acids selected from the amino acid sequence of SEQ ID NO: 71, including SEQ ID NO: 73 (EEPSQ) as an essential element. For example, the c-Met inhibitor may specifically bind to an epitope including the amino acid sequence of SEQ ID NO: 71, SEQ ID NO: 72, or SEQ ID NO: 73.

In one embodiment, the c-Met inhibitor or an antigen-binding fragment thereof may comprise or consist essentially of:

at least one heavy chain complementarity determining region (CDR) selected from the group consisting of (a) a CDR-H1 comprising the amino acid sequence of SEQ ID NO: 4; (b) a CDR-H2 comprising the amino acid sequence of SEQ ID NO: 5, SEQ ID NO: 2, or an amino acid sequence comprising 8-19 consecutive amino acids within SEQ ID NO: 2 including amino acid residues from the 3^{rd} to 10^{th} positions of SEQ ID NO: 2; and (c) a CDR-H3 comprising the amino acid sequence of SEQ ID NO: 6, SEQ ID NO: 85, or an amino acid sequence comprising 6-13 consecutive amino acids within SEQ ID NO: 85 including amino acid residues from the 1^{st} to 6^{th} positions of SEQ ID NO: 85, or a heavy chain variable region comprising the at least one heavy chain complementarity determining region;

at least one light chain complementarity determining region (CDR) selected from the group consisting of (a) a CDR-L1 comprising the amino acid sequence of SEQ ID NO: 7, (b) a CDR-L2 comprising the amino acid sequence of SEQ ID NO: 8, and (c) a CDR-L3 comprising the amino acid sequence of SEQ ID NO: 9, SEQ ID NO: 15, SEQ ID NO: 86, or an amino acid sequence comprising 9-17 consecutive amino acids within SEQ ID NO: 89 including amino acid residues from the 1^{st} to 9^{th} positions of SEQ ID NO: 89, or a light chain variable region comprising the at least one light chain complementarity determining region;

a combination of the at least one heavy chain complementarity determining region and at least one light chain complementarity determining region; or

a combination of the heavy chain variable region and the light chain variable region.

Herein, the amino acid sequences of SEQ ID NOS: 4 to 9 are respectively represented by following Formulas I to VI, below:

| | |
|---|---|
| Formula I | |
| Xaa₁-Xaa₂-Tyr-Tyr-Met-Ser, | (SEQ ID NO: 4) |

wherein Xaa₁ is absent or Pro or Ser, and Xaa₂ is Glu or Asp,

| | |
|---|---|
| Formula II | |
| Arg-Asn-Xaa₃-Xaa₄-Asn-Gly-Xaa₅-Thr, | (SEQ ID NO: 5) |

wherein Xaa₃ is Asn or Lys, Xaa₄ is Ala or Val, and Xaa₅ is Asn or Thr,

| Formula III | |
|---|---|
| Asp-Asn-Trp-Leu-Xaa₆-Tyr, | (SEQ ID NO: 6) |

wherein Xaa₆ is Ser or Thr,

Formula IV
(SEQ ID NO: 7)
Lys-Ser-Ser-Xaa₇-Ser-Leu-Leu-Ala-Xaa₈-Gly-Asn-Xaa₉-
Xaa₁₀-Asn-Tyr-Leu-Ala

wherein Xaa₇ is His, Arg, Gln, or Lys, Xaa₈ is Ser or Trp, Xaa₉ is His or Gln, and Xaa₁₀ is Lys or Asn,

| Formula V | |
|---|---|
| Trp-Xaa₁₁-Ser-Xaa₁₂-Arg-Val-Xaa₁₃ | (SEQ ID NO: 8) |

wherein Xaa₁₁ is Ala or Gly, Xaa₁₂ is Thr or Lys, and Xaa₁₃ is Ser or Pro, and

Formula VI
(SEQ ID NO: 9)
Xaa₁₄-Gln-Ser-Tyr-Ser-Xaa₁₅-Pro-Xaa₁₆-Thr

wherein Xaa₁₄ is Gly, Ala, or Gln, Xaa₁₅ is Arg, His, Ser, Ala, Gly, or Lys, and Xaa₁₆ is Leu, Tyr, Phe, or Met.

In one embodiment, the CDR-H1 may comprise or consist essentially of an amino acid sequence selected from the group consisting of SEQ ID NOS: 1, 22, 23, and 24. The CDR-H2 may comprise or consist essentially of an amino acid sequence selected from the group consisting of SEQ ID NOS: 2, 25, and 26. The CDR-H3 may comprise or consist essentially of an amino acid sequence selected from the group consisting of SEQ ID NOS: 3, 27, 28, and 85.

The CDR-L1 may comprise or consist essentially of an amino acid sequence selected from the group consisting of SEQ ID NOS: 10, 29, 30, 31, 32, 33, and 106. The CDR-L2 may comprise or consist essentially of an amino acid sequence selected from the group consisting of SEQ ID NOS: 11, 34, 35, and 36. The CDR-L3 may comprise or consist essentially of an amino acid sequence selected from the group consisting of SEQ ID NOS: 12, 13, 14, 15, 16, 37, 86, and 89.

In another embodiment, the antibody or antigen-binding fragment may comprise:

a heavy chain variable region comprising a polypeptide (CDR-H1) comprising or consisting essentially of an amino acid sequence selected from the group consisting of SEQ ID NOS: 1, 22, 23, and 24, a polypeptide (CDR-H2) comprising or consisting essentially of an amino acid sequence selected from the group consisting of SEQ ID NOS: 2, 25, and 26, and a polypeptide (CDR-H3) comprising or consisting essentially of an amino acid sequence selected from the group consisting of SEQ ID NOS: 3, 27, 28, and 85;

a light chain variable region comprising a polypeptide (CDR-L1) comprising or consisting essentially of an amino acid sequence selected from the group consisting of SEQ ID NOS: 10, 29, 30, 31, 32, 33 and 106, a polypeptide (CDR-L2) comprising or consisting essentially of an amino acid sequence selected from the group consisting of SEQ ID NOS: 11, 34, 35, and 36, and a polypeptide (CDR-L3) comprising or consisting essentially of an amino acid sequence selected from the group consisting of SEQ ID NOS 12, 13, 14, 15, 16, 37, 86, and 89; or

a combination of the heavy chain variable region and the light chain variable region.

In one embodiment, the anti-c-Met antibody or antigen-binding fragment thereof may comprise:

a variable region of the heavy chain comprising or consisting essentially of the amino acid sequence of SEQ ID NO: 17, 74, 87, 90, 91, 92, 93, or 94,

a variable region of the light chain comprising or consisting essentially of the amino acid sequence of SEQ ID NO: 134, 18, 19, 20, 21, 75, 88, 95, 96, 97, 98, 99, or 107; or

a combination thereof.

In one embodiment, the anti-c-Met antibody may be a monoclonal antibody. The monoclonal antibody may be produced by the hybridoma cell line deposited with Accession No. KCLRF-BP-00220, which binds specifically to the extracellular region of c-Met protein (refer to Korean Patent Publication No. 2011-0047698, the disclosure of which is incorporated in its entirety herein by reference). The anti-c-Met antibody may include all the antibodies defined in Korean Patent Publication No. 2011-0047698.

By way of further example, the anti-c-Met antibody or the antibody fragment may comprise or consist essentially of:

a heavy chain including the amino acid sequence selected from the group consisting of the amino acid sequence of SEQ ID NO: 62 (wherein the amino acid sequence from amino acid residues from the 1^{st} to 17^{th} positions is a signal peptide), or the amino acid sequence from the 18^{th} to 462^{nd} positions of SEQ ID NO: 62, the amino acid sequence of SEQ ID NO: 64 (wherein the amino acid sequence from the 1^{st} to 17^{th} positions is a signal peptide), the amino acid sequence from the 18^{th} to 461^{st} positions of SEQ ID NO: 64, the amino acid sequence of SEQ ID NO: 66 (wherein the amino acid sequence from the 1^{st} to 17^{th} positions is a signal peptide), and the amino acid sequence from the 18^{th} to 460^{th} positions of SEQ ID NO: 66; and

a light chain including the amino acid sequence selected from the group consisting of the amino acid sequence of SEQ ID NO: 68 (wherein the amino acid sequence from the 1^{st} to 20^{th} positions is a signal peptide), the amino acid sequence from the 21^{st} to 240^{th} positions of SEQ ID NO: 68, the amino acid sequence of SEQ ID NO: 70 (wherein the amino acid sequence from the 1^{st} to 20^{th} positions is a signal peptide), the amino acid sequence from the 21^{st} to 240^{th} positions of SEQ ID NO: 70, and the amino acid sequence of SEQ ID NO: 108.

For example, the anti-c-Met antibody may be selected from the group consisting of:

an antibody including a heavy chain including the amino acid sequence of SEQ ID NO: 62 or the amino acid sequence from the 18^{th} to 462^{nd} positions of SEQ ID NO: 62 and a light chain including the amino acid sequence of SEQ ID NO: 68 or the amino acid sequence from the 21^{st} to 240^{th} positions of SEQ ID NO: 68;

an antibody including a heavy chain including the amino acid sequence of SEQ ID NO: 64 or the amino acid sequence from the 18^{th} to 461^{st} positions of SEQ ID NO: 64 and a light chain including the amino acid sequence of SEQ ID NO: 68 or the amino acid sequence from the 21^{st} to 240^{th} positions of SEQ ID NO: 68;

an antibody including a heavy chain including the amino acid sequence of SEQ ID NO: 66 or the amino acid sequence from the 18^{th} to 460^{th} positions of SEQ ID NO: 66 and a light chain including the amino acid sequence of SEQ ID NO: 68 or the amino acid sequence from the 21^{st} to 240^{th} positions of SEQ ID NO: 68;

an antibody including a heavy chain including the amino acid sequence of SEQ ID NO: 62 or the amino acid sequence from the 18^{th} to 462^{nd} positions of SEQ ID NO: 62 and a light chain including the amino acid sequence of SEQ ID NO: 70 or the amino acid sequence from the 21^{st} to 240^{th} positions of SEQ ID NO: 70;

an antibody including a heavy chain including the amino acid sequence of SEQ ID NO: 64 or the amino acid sequence from the 18^{th} to 461^{st} positions of SEQ ID NO: 64 and a light chain including the amino acid sequence of SEQ ID NO: 70 or the amino acid sequence from the 21^{st} to 240^{th} positions of SEQ ID NO: 70;

an antibody including a heavy chain including the amino acid sequence of SEQ ID NO: 66 or the amino acid sequence from the 18^{th} to 460^{th} positions of SEQ ID NO: 66 and a light chain including the amino acid sequence of SEQ ID NO: 70 or the amino acid sequence from the 21^{st} to 240^{th} positions of SEQ ID NO: 70;

an antibody including a heavy chain including the amino acid sequence of SEQ ID NO: 62 or the amino acid sequence from the 18^{th} to 462^{nd} positions of SEQ ID NO: 62 and a light chain including the amino acid sequence of SEQ ID NO: 108;

an antibody including a heavy chain including the amino acid sequence of SEQ ID NO: 64 or the amino acid sequence from the 18^{th} to 461^{st} positions of SEQ ID NO: 64 and a light chain including the amino acid sequence of SEQ ID NO: 108; and

an antibody including a heavy chain including the amino acid sequence of SEQ ID NO: 66 or the amino acid sequence from the 18^{th} to 460^{th} positions of SEQ ID NO: 66 and a light chain including the amino acid sequence of SEQ ID NO: 108.

In a particular embodiment, the anti-c-Met antibody may be an antibody comprising a heavy chain comprising or consisting essentially of the amino acid sequence of SEQ ID NO: 66 or the amino acid sequence from the 18^{th} to 460^{th} positions of SEQ ID NO: 66 and a light chain comprising or consisting essentially of the amino acid sequence of SEQ ID NO: 68 or the amino acid sequence from the 21^{st} to 240^{th} positions of SEQ ID NO: 68.

The polypeptide of SEQ ID NO: 70 is a light chain including human kappa (κ) constant region, and the polypeptide with the amino acid sequence of SEQ ID NO: 68 is a polypeptide obtained by replacing histidine at position 62 (corresponding to position 36 of SEQ ID NO: 68 according to kabat numbering) of the polypeptide with the amino acid sequence of SEQ ID NO: 70 with tyrosine. The production yield of the antibodies may be increased by the replacement. The polypeptide with the amino acid sequence of SEQ ID NO: 108 is a polypeptide obtained by replacing serine at position 32 (position 27e according to kabat numbering in the amino acid sequence from amino acid residues 21 to 240 of SEQ ID NO: 68; positioned within CDR-L1) with tryptophan. By such replacement, antibodies and antibody fragments including such sequences exhibits increased activities, such as c-Met biding affinity, c-Met degradation activity, and Akt phosphorylation inhibition.

The anti-c-Met antibodies may be, but not limited to, animal antibodies (e.g., mouse-derived antibodies), chimeric antibodies (e.g., mouse-human chimeric antibodies), humanized antibodies, or human antibodies. The antibodies or antigen-binding fragments thereof may be isolated from a living body or non-naturally occurring. The antibodies or antigen-binding fragments thereof may be synthetic or recombinant. The antibody may be monoclonal.

In one embodiment, the anti-c-Met antibody or an antigen-binding fragment thereof may be modified by any combination of deletion, insertion, addition, or substitution of at least one amino acid residue on the amino acid sequence of the hinge region so that it exhibit enhanced antigen-binding efficiency. For example, the antibody may include a hinge region including the amino acid sequence of SEQ ID NO: 100(U7-HC6), 101(U6-HC7), 102(U3-HC9), 103(U6-HC8), or 104(U8-HC5), or a hinge region including the amino acid sequence of SEQ ID NO: 105 (non-modified human hinge). In particular, the hinge region has the amino acid sequence of SEQ ID NO: 100 or 101.

In the c-Met antibody or an antigen-binding fragment thereof, the rest of the light chain and the heavy chain portion except the CDRs, the light chain variable region, and the heavy chain variable region as defined above, for example, the light chain constant region and the heavy chain constant region, may be from any subtype of immunoglobulin (e.g., IgA, IgD, IgE, IgG (IgG1, IgG2, IgG3, IgG4), IgM, and the like).

The term “antigen-binding fragment” used herein refers to fragments of an intact immunoglobulin including portions of a polypeptide including antigen-binding regions having the ability to specifically bind to the antigen. In a particular embodiment, the antigen-binding fragment may be scFv, (scFv)₂, scFvFc, Fab, Fab′, or F(ab′)₂, but is not limited thereto.

When the polypeptide of interest is an anti-c-Met antibody or an antigen-binding fragment thereof, a gene of interest may be at least one selected from the group consisting of a polynucleotide encoding a heavy chain CDR or a light chain CDR, a polynucleotide encoding a heavy chain variable region or a light chain variable region, and a polynucleotide encoding a heavy chain or a light chain, wherein the CDRs, variable regions, a heavy chain and a light chain are as described above. For example, the gene of interest may be at least one selected from the group consisting of SEQ ID NO: 38, SEQ ID NO: 39, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 55, SEQ ID NO: 58, SEQ ID NO: 59, SEQ ID NO: 60, SEQ ID NO: 61, SEQ ID NO: 63, SEQ ID NO: 65, SEQ ID NO: 67, SEQ ID NO: 69, SEQ ID NO: 76, and SEQ ID NO: 77.

In another embodiment, provided is a polynucleotide comprising or consisting essentially of the nucleotide sequence of SEQ ID NO: 124. The polynucleotide of SEQ ID NO: 124 may be useful as a promoter which can operate in an animal cell, such as a mammalian cell. In another embodiment, provided is a recombinant vector comprising the nucleotide sequence of SEQ ID NO: 124. In another embodiment, provide is a recombinant cell comprising (transfected with) the recombinant vector. The recombinant vector and the recombinant cell are as described above.

This disclosure may provide a recombinant vector for an animal cell (e.g., a mammalian cell) for high expression of a therapeutic protein or antibody, which can be useful in mass-production of various therapeutic proteins such as anti-c-Met antibodies.

### EXAMPLES

Hereafter, the present invention will be described in detail by examples.

The following examples are intended merely to illustrate the invention and are not construed to restrict the invention.

### Example 1

### Preparation of a Recombinant Vector

In this example, the expression of a protein of interest under the control of human CMV (hCMV) promoter and a fusion promoter comprising a combination of hCMV promoter and intron were compared to each other.

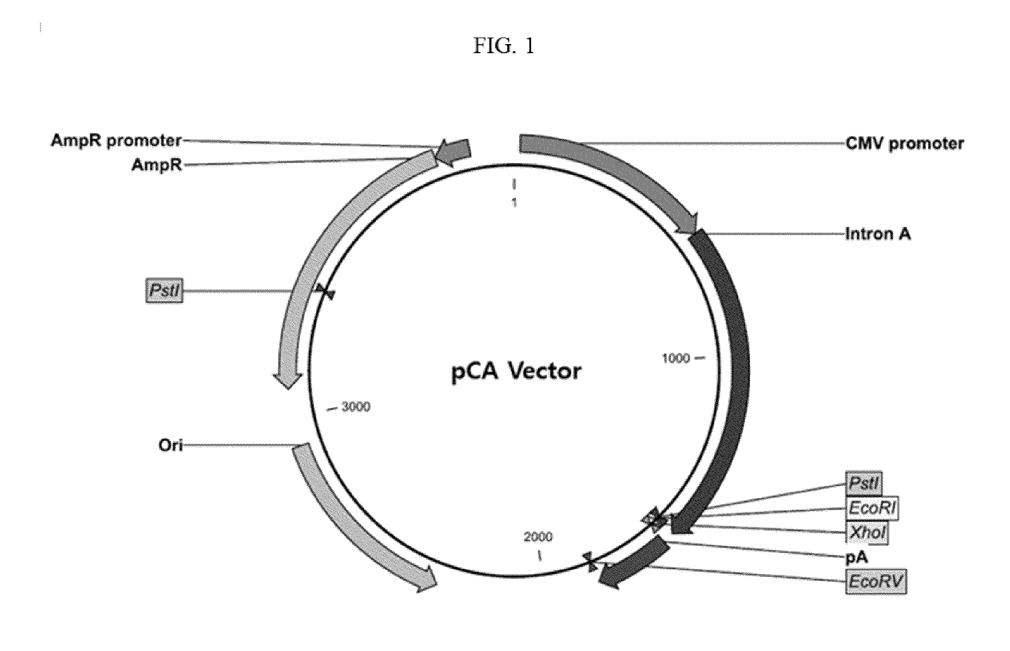

A fusion promoter of hCMV promoter (SEQ ID NO: 124; at 5′ end) and intron A (SEQ ID NO: 113; at 3′ end) was synthesized, and based thereon, a basic vector pCA (see, FIG. 1) was constructed as shown in FIG. 1.

For combinations of hCMV promoter and other introns than intron A, a hCMV promoter fragment having MfeI restriction site at 3′ end was amplified by PCR. Using a forward primer (CA-Fw), a reverse primer having MfeI restriction site at 3′ end, and pCA vector of FIG. 1 as a template, a PCR was performed by 20 cycles under the conditions of 94° C. and 5 minutes, 94° C. and 30 seconds, 55° C. and 30 seconds, and 72° C. and 1 minute, and then, elongation under the conditions of 72° C. and 5 minutes, to obtain a hCMV promoter fragment. The primers used in PCR are summarized in Table 3.

In addition, the intron genes used in the combination of hCMV promoter and intron were summarized in Table 2:

TABLE 2

| | | SEQ |
|---|---|---|
| | | ID |
| Intron | Sequence of Intron gene | NO: |
| IGLV-1L1 Int | gtgacaggat ggggaccaag aaaggggccc tgggaagccc atggggccct gctttctcct cttgtctcct | 109 |
| (115 bP) | tttgtctctt gtcaatcacc atgtctgtgt ctctctcact tccag | |
| IGKV Int | gtgagaatatttagaaaaagctaaaactaattctttgaaccattaattttcttaattaggaacctggcaccatatggaac | 110 |
| (206 bp) | ttggcttgtttttaaatgtgtttttttttaagtaatgcgtattctttcatcttgtgctactagattagtggtgatttcattaagc | |
| | agatgcttatattgtgctaatgtttgctgtatgttttcag | |
| IGH Int | gtgagtgtctcagggatccagacatgggggtatgggaggtgcctctgatcccagggctcactgtgggtctctctgtt | 111 |
| (82 bp) | cacag | |
| Chimeric | GTAAGTATCAAGGTTACAAGACAGGTTTAAGGAGACCAATAGAAAC | 112 |
| Intron | TGGGCTTGTCGAGACAGAGAAGACTCTTGCGTTTCTGATAGGCACCT | |
| (133 bp) | ATTGGTCTTACTGACATCCACTTTGCCTTTCTCTCCACAG | |
| Intron A | gtttagtgaaccgtcagatcgcctggagacgccatccacgctgttttgacctccatagaagacaccgggaccgatc | 113 |
| (963 bp) | cagcctccgcggccgggaacggtgcattggaacgcggattccccgtgccaagagtgacgtaagtaccgcctata | |
| | gactctataggcacacccctttggctcttatgcatgctatactgtttttggcttggggcctatacacccccgctccttat | |
| | gctataggtgatggtatagcttagcctataggtgtgggttattgaccattattgaccactcccctattggtgacgatact | |
| | ttccattactaatccataacatggctctttgccacaactatctctattggctatatgccaatactctgtccttcagagact | |
| | gacacggactctgtatttttacaggatggggtcccatttattatttacaaattcacatatacaacaacgccgtcccccgt | |
| | gcccgcagtttttattaaacatagcgtgggatctccacgcgaatctcgggtacgtgttccggacatgggctcttctcc | |
| | ggtagcggcggagcttccacatccgagccctggtcccatgcctccagcggctcatggtcgctcggcagctccttg | |
| | ctcctaacagtggaggccagacttaggcacagcacaatgcccaccaccaccagtgtgccgcacaaggccgtgg | |
| | cggtagggtatgtgtctgaaaatgagctcggagattgggctcgcaccgtgacgcagatggaagacttaaggcag | |
| | cggcagaagaagatgcaggcagctgagttgttgtattctgataagagtcagaggtaactcccgttgcggtgctgtta | |
| | acggtggagggcagtgtagtctgagcagtactcgttgctgccgcgcgcgccaccagacataatagctgacagac | |
| | taacagactgttcctttccatgggtcttttctgcagtcacc | |

TABLE 3
Primers used in PCR

| SEQ | Oligomer | |
|---|---|---|
| ID No. | Name | Sequence |
| 127 | CA-Fw | 5′-TAACAGGGTAATATAG acgcgtgga-3′ |
| 128 | hCMV-Rv | 5′-CAATTG agagctctgcttat-3′ |
| 129 | LInt-Fw | 5′-agagctct CAATTG gtgacagga-3′ |
| 130 | KInt-Fw | 5′-agagctct CAATTG gt gagaatat-3′ |
| 131 | HInt-Fw | 5′-agagctct CAATTG gtga gtgtct-3′ |
| 132 | CInt-Fw | 5′-agagctct CAATTG GTAAGTATC-3′ |
| 133 | CA-Rev | 5′-ttctcgagttctccgctagctcct-3′ |

Each of the above introns was prepared by genetic synthesis. For preparing a fusion promoter comprising a hCMV promoter and each intron, a PCR (94° C. and 5 minutes, 94° C. and 30 seconds, 55° C. and 30 seconds, and 72° C. and 40 seconds; 20 cycles, and elongation at 72° C. for 5 minutes) was performed using primers (LInt-Fw, Klnt-Fw, HInt-Fw, and CInt-Fw: see Table 3) having MfeI restriction site (5′-CAATTG-3′) at 5′ end and a reverse primer (CA-Rv: SEQ ID NO: 133) having EcoRI restriction site (5′-GAATTC-3′) at 3′ end, to amplify each intron fragment.

Using the hCMV promoter and intron fragments obtained by each PCR as a template and using a 5′-end forward primer of hCMV promoter (CA-Fw) and 3′-end reverse primer of intron fragment (CA-Rv), a second PCR (94° C. and 5 minutes, 94° C. and 30 seconds, 55° C. and 30 seconds, and 72° C. and 1 minute; 20 cycles, and elongation at 72° C. for 5 minutes) was performed to obtain fusion promoters each of which comprises a combination of the hCMV promoter and each intron. Each of the obtained fusion promoter fragments was cloned into vector pCA (FIG. 1) which is pre-restricted with MluI/EcoRI (New England Biolabs), to construct a vector comprising a combination of the hCMV promoter and each intron.

To produce an anti-c-Met antibody as a protein of interest, a heavy chain coding polynucleotide (SEQ ID NO: 67) and a light chain coding polynucleotide (SEQ ID NO: 69) were respectively cloned into each of the constructed vectors using restriction enzymes, EcoRI and XhoI, to construct a recombinant vector for a heavy chain and a recombinant vector for a light chain of an anti-c-Met antibody.

### Example 2

### Measurement of Protein Expression Level by a Recombinant Vector

Each of the recombinant vectors constructed in Example 1 was isolated using Qiagen EndoFree Plasmid Mega kit (Cat no. 12381), and subjected to a transient transfection into a mammalian cell. The amount of the protein (antibody), which is expressed from the anti-c-Met antibody gene cloned in the vectors and secreted, was measured and compared to that of a control.

### Example 2-1

### Protein Expression in Transfected HEK293 Cells

The vectors constructed in Example 1 were transfected into HEK293-F cells and Expi293 cells, respectively. The cell lines HEK293-F and Expi293 were purchased from Invitrogen, and cultured by suspension culture using FreeStyle™ 293 Expression Medium (Invitrogen) and Expi293™ Expression Medium (Invitrogen), respectively. Each cell line was seeded at the concentration of 2×10⁵ cells/mL, raised, and segmented every 4 days.

To measure the efficacy of the vectors, on one day before transient gene expression, the cells were provided at the concentration of 5×10⁵ cells/mL, and 24 hours after, when the cell number reached 1×10⁶ cells/mL, a transfection was performed. In case of 293-F cells, 90 mL of 293-F cells (cell concentration: 1×10⁶ cells/mL) were provided in 250 mL Erlenmeyer flask, and subjected to a transfection by liposomal reagent method using Freestyle™ MAX reagent (Invitrogen). Each of the recombinant vectors constructed in Example 1 were provided in a 15 ml tube so that the ratio of heavy chain DNA: light chain DNA reaches 1:1, and mixed with 2 ml of OptiPro™ SFM (Invitrogen) (tube A). A mixture of 100 μl of Freestyle™ MAX reagent and 2 ml OptiPro™ SFM was provided in another 15 ml tube (tube B). The tube A and tube B was mixed and incubated for 15 minutes, and the mixture solution was slowly dropped onto the provided cells to transfect the cells with the vectors. After completing the transfection, the transfected cells were incubated in an incubator under the conditions of 37° C., 80% humidity, 8% CO₂, and 130 rpm, for 5 days. Then, the supernatant was collected and the concentration of obtained anti-c-Met antibody therein was measured using a Protein A biosensor of Octet system (ForteBio). For comparison, the same experiment was performed using a vector with only hCMV (SEQ ID NO: 123; derived from pcDNA 3.3 TOPO vector (Invitrogen)) as a promoter (a control).

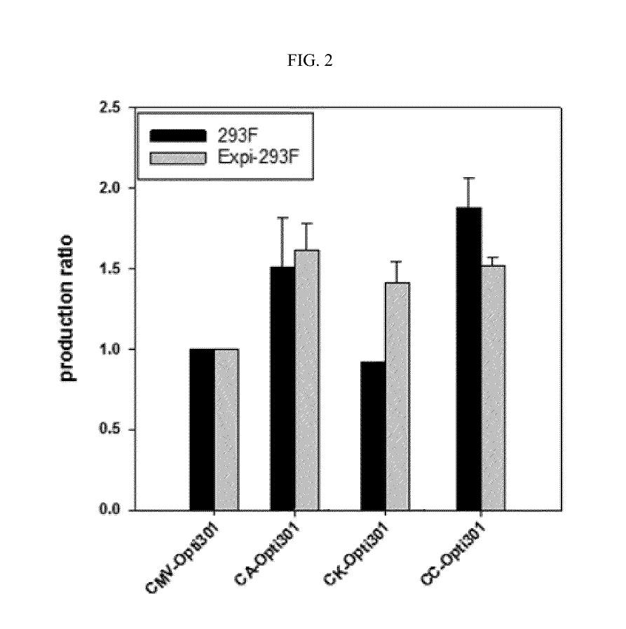

The measured antibody concentrations obtained using each fusion promoter are shown in FIG. 2, wherein the concentrations were indicated as a ratio to that of the control (CMV-Opti301 (a control): human CMV promoter only, CA-Opti301: human CMV promoter+Intron A, CK-Opti301: human CMV promoter+IGKV Intron, CC-Opti301: human CMV promoter+chimeric intron). As shown in FIG. 2, antibody production level is considerably increased compared to control using the fusion promoter whereby hCMV of SEQ ID NO: 124 is fused with one of the various introns.

### Example 2-2

### Protein Expression in Transfected CHO-S Cells

A CHO-S cell line (Invitrogen) is a mutant of a CHO-K1 cell line. The CHO-S cell line was cultured and raised by suspension culture using FreeStyle™ CHO expression medium containing 8 mM glutamine, and seeded at the concentration of 2×10⁵ cells/mL, and segmented every 4 days. The cells were cultured under the conditions of 36.5° C., 5% CO₂, 80% humidity, and 130 rpm. On one day before transient gene expression, the cells were provided at the concentration of 5×10⁵ cells/mL, and 24 hours after, when the cell number reached 1×10⁶ cells/mL, a transfection was performed.

Referring to the method described in Example 2-1, transfection was performed using each of the recombinant vector constructs in Example 1. Five days after transfection, the supernatant was collected and the concentration of the anti-c-Met antibody was measured using a Protein A biosensor of Octet system (ForteBio). For comparison, a same experiment was performed using a vector having hCMV promoter (SEQ ID NO: 123; derived from pcDNA 3.3 TOPO vector (Invitrogen)) only as a promoter (a control).

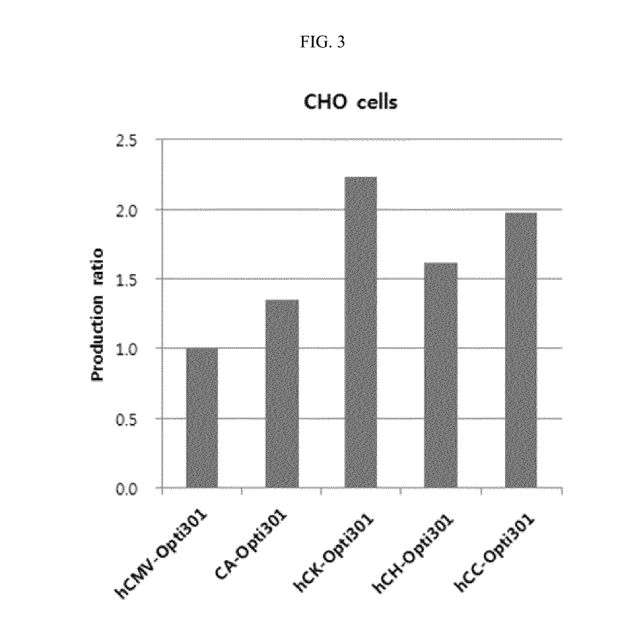

The measured antibody concentrations obtained using each fusion promoter were shown in FIG. 3, wherein the concentrations were indicated as a ratio to that of the control (hCMV-Opti301 (control): human CMV promoter only, CA-Opti301: human CMV promoter+Intron A, hCK-Opti301: human CMV promoter+IGKV Intron, hCH-Opti301: human CMV promoter+IGH Intron, hCC-Opti301: human CMV promoter+chimeric intron). As shown in FIG. 3, when the fusion promoter where hCMV promoter of SEQ ID NO: 124 is fused with one of various introns, the antibody production level is considerably increased, compared with the control.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein. The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A fusion polynucleotide comprising a human CMV promoter and an immunoglobulin intron.

2. The fusion polynucleotide of claim 1, wherein the human CMV promoter is:
i) a polynucleotide fragment comprising SEQ ID NO: 116,
ii) a polynucleotide fragment comprising consecutive nucleotide residues of at least 100 bp within a region from position 400 to position 1250 of SEQ ID NO: 116,
iii-1) polynucleotide variant comprising at least one mutation selected from the group consisting of
a) a substitution of at least one nucleotide in the polynucleotide fragment i) or ii), wherein the at least one nucleotide is selected from the group consisting of nucleotides corresponding to positions 490 (C), 529 (C), 532 (T), 545 (A), 504 (A), 651 (G), 804 (A), 870 (T), 946 (T), 1061 (C), 1065 (T), and 1073 (A) of SEQ ID NO: 116,
b) a deletion of a nucleotide of the polynucleotide fragment i) or ii) corresponding to the position 653 of SEQ ID NO: 116, and
c) a insertion of a nucleotide between the nucleotides of the polynucleotide fragment i) or ii) corresponding to the positions 805 and 806 of SEQ ID NO: 116, or
iii-2) a polynucleotide variant further comprising nucleotides of 1 to 100 bp at 5′-end, 3′-end, or both ends of the polynucleotide fragment i) or ii) or the polynucleotide variant iii-1).

3. The fusion polynucleotide of claim 1, wherein the human CMV promoter is:
i) a polynucleotide fragment comprising SEQ ID NO: 116,
ii) a polynucleotide fragment comprising at least 400 consecutive nucleotide residues of SEQ ID NO: 116 in a 3′-terminal direction starting from position 400 to position 410, position 530 to position 550, or position 615 to position 625 of SEQ ID NO: 116,
iii-1) a polynucleotide variant comprising at least one mutation selected from the group consisting of:
a substitution of nucleotide C of the polynucleotide fragment i) or ii) corresponding to the position 490 of SEQ ID NO: 116 with T (C490T),
a substitution of nucleotide T of the polynucleotide fragment i) or ii) corresponding to the position 532 of SEQ ID NO: 116 with G (T532G),
a substitution of nucleotide A of the polynucleotide fragment i) or ii) corresponding to the position 545 of SEQ ID NO: 116 with G (A545G),
a substitution of nucleotide G of the polynucleotide fragment i) or ii) corresponding to the position 651 of SEQ ID NO: 116 with C (G651C),
a substitution of nucleotide A of the polynucleotide fragment i) or ii) corresponding to the position 804 of SEQ ID NO: 116 with C (A804C),
a substitution of nucleotide T of the polynucleotide fragment i) or ii) corresponding to the position 870 of SEQ ID NO: 116 with C (T870C),
a substitution of nucleotide T of the polynucleotide fragment i) or ii) corresponding to the position 946 of SEQ ID NO: 116 with C (T946C),
a substitution of nucleotide C of the polynucleotide fragment i) or ii) corresponding to the position 1061 of SEQ ID NO: 116 with T (C1061T),
a substitution of nucleotide T of the polynucleotide fragment i) or ii) corresponding to the position 1065 of SEQ ID NO: 116 with C (T1065C),
a substitution of nucleotide A of the polynucleotide fragment i) or ii) corresponding to the position 1073 of SEQ ID NO: 116 with G (A1073G),
a deletion of a nucleotide C of the polynucleotide fragment i) or ii) corresponding to the position 653 of SEQ ID NO: 116, and
a insertion of a nucleotide between the nucleotides of the polynucleotide fragment i) or ii) corresponding to the positions 805 and 806 of SEQ ID NO: 116, or
iii-2) a polynucleotide variant further comprising nucleotides of 5 to 20 bp or 45 to 60 bp at 3′-end of the polynucleotide fragment i) or ii) or the polynucleotide variant iii-1).

4. The fusion polynucleotide of claim 3, wherein the human CMV promoter comprises one of SEQ ID NOs: 117 to 123.

5. The fusion polynucleotide of claim 1, wherein the immunoglobulin intron is at least one selected from the group consisting of an IGLV intron, an IGKV intron, and an IGH intron.

6. The fusion polynucleotide of claim 5, wherein the immunoglobulin intron comprises at least one selected from SEQ ID NOs: 109 to 111.

7. The fusion polynucleotide of claim 1, wherein the immunoglobulin intron is linked to 3′-end of the human CMV promoter.

8. An expression vector comprising the fusion polynucleotide of claim 1.

9. A fusion polynucleotide comprising a human CMV promoter and an intron, wherein the human CMV promoter is 1) a polynucleotide comprising SEQ ID NO: 124, 2) a polynucleotide comprising at least 200 consecutive nucleotides of SEQ ID NO: 124, or 3) a polynucleotide comprising 1) or 2) and further comprising 1-100 additional nucleotides at 3′-end, 5′-end or both ends of the polynucleotide.

10. The fusion polynucleotide of claim 9, wherein the intron is at least one selected from the group consisting of an immunoglobulin intron, a chimeric intron, and an intron A of human cytomegalovirus major immediate-early release protein gene.

11. The fusion polynucleotide of claim 10, wherein the immunoglobulin intron is at least one selected from the group consisting of an IGLV intron, an IGKV intron, and an IGH intron.

12. The fusion polynucleotide of claim 10, wherein the immunoglobulin intron comprises at least one selected from SEQ ID NOs: 109 to 111.

13. The fusion polynucleotide of claim 9, wherein the immunoglobulin intron is linked to 3′-end of the human CMV promoter.

14. An expression vector comprising the fusion polynucleotide of claim 9.

15. A recombinant vector comprising the fusion polynucleotide of claim 1.

16. The recombinant vector of claim 15, further comprising a gene encoding a polypeptide of interest operatively linked to the fusion polynucleotide.

17. A recombinant vector comprising the fusion polynucleotide of claim 9.

18. The recombinant vector of claim 17, further comprising a gene encoding a polypeptide of interest operatively linked to the fusion polypeptide.

19. A recombinant cell comprising the recombinant vector of claim 16.

20. The recombinant cell of claim 19, wherein the cell is a mammalian cell.

21. A recombinant cell comprising the recombinant vector of claim 18.

22. The recombinant cell of claim 21, wherein the cell is a mammalian cell.

23. A method of producing a polypeptide of interest, comprising expressing the gene encoding the polypeptide of interest in the recombinant cell of claim 19.

24. A method of producing a polypeptide of interest, comprising expressing the gene encoding the polypeptide of interest in the recombinant cell of claim 21.


SEQUENCE LISTING (134 sequences)

SEQ ID NO: 1 - protein, 5 aa, Artificial Sequence
  Synthetic heavy chain CDR1 of AbF46
DYYMS

SEQ ID NO: 2 - protein, 19 aa, Artificial Sequence
  Synthetic heavy chain CDR2 of AbF46
FIRNKANGYTTEYSASVKG

SEQ ID NO: 3 - protein, 6 aa, Artificial Sequence
  Synthetic heavy chain CDR3 of AbF46
DNWFAY

SEQ ID NO: 4 - protein, 6 aa, Artificial Sequence
  Synthetic heavy chain CDR1 of c-Met antibody
XXYYMS

SEQ ID NO: 5 - protein, 8 aa, Artificial Sequence
  Synthetic heavy chain CDR2 of c-Met antibody
RNXXNGXT

SEQ ID NO: 6 - protein, 6 aa, Artificial Sequence
  Synthetic heavy chain CDR3 of c-Met antibody
DNWLXY

SEQ ID NO: 7 - protein, 17 aa, Artificial Sequence
  Synthetic light chain CDR1 of c-Met antibody
KSSXSLLAXGNXXNYLA

SEQ ID NO: 8 - protein, 7 aa, Artificial Sequence
  Synthetic light chain CDR2 of c-Met antibody
WXSXRVX

SEQ ID NO: 9 - protein, 9 aa, Artificial Sequence
  Synthetic light chain CDR3 of c-Met antibody
XQSYSXPXT

SEQ ID NO: 10 - protein, 17 aa, Artificial Sequence
  Synthetic light chain CDR1 of AbF46
KSSQSLLASGNQNNYLA

SEQ ID NO: 11 - protein, 7 aa, Artificial Sequence
  Synthetic light chain CDR2 of AbF46
WASTRVS

SEQ ID NO: 12 - protein, 9 aa, Artificial Sequence
  Synthetic light chain CDR3 of AbF46
QQSYSAPLT

SEQ ID NO: 13 - protein, 9 aa, Artificial Sequence
  Synthetic CDR-L3 derived from L3-1 clone
QQSYSRPYT

SEQ ID NO: 14 - protein, 9 aa, Artificial Sequence
  Synthetic CDR-L3 derived from L3-2 clone
GQSYSRPLT

SEQ ID NO: 15 - protein, 9 aa, Artificial Sequence
  Synthetic CDR-L3 derived from L3-3 clone
AQSYSHPFS

SEQ ID NO: 16 - protein, 9 aa, Artificial Sequence
  Synthetic CDR-L3 derived from L3-5 clone
QQSYSRPFT

SEQ ID NO: 17 - protein, 117 aa, Artificial Sequence
  Synthetic heavy chain variable region of anti c-Met humanized antibody(huAbF46-H4)
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSS

SEQ ID NO: 18 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKR

SEQ ID NO: 19 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCGQSYSRPLTFGQGTKVEIKR

SEQ ID NO: 20 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCAQSYSHPFSFGQGTKVEIKR

SEQ ID NO: 21 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPFTFGQGTKVEIKR

SEQ ID NO: 22 - protein, 6 aa, Artificial Sequence
  Synthetic CDR-H1 derived from H11-4 clone
PEYYMS

SEQ ID NO: 23 - protein, 6 aa, Artificial Sequence
  Synthetic CDR-H1 derived from YC151 clone
PDYYMS

SEQ ID NO: 24 - protein, 6 aa, Artificial Sequence
  Synthetic CDR-H1 derived from YC193 clone
SDYYMS

SEQ ID NO: 25 - protein, 8 aa, Artificial Sequence
  Synthetic CDR-H2 derived from YC244 clone
RNNANGNT

SEQ ID NO: 26 - protein, 8 aa, Artificial Sequence
  Synthetic CDR-H2 derived from YC321 clone
RNKVNGYT

SEQ ID NO: 27 - protein, 6 aa, Artificial Sequence
  Synthetic CDR-H3 derived from YC354 clone
DNWLSY

SEQ ID NO: 28 - protein, 6 aa, Artificial Sequence
  Synthetic CDR-H3 derived from YC374 clone
DNWLTY

SEQ ID NO: 29 - protein, 17 aa, Artificial Sequence
  Synthetic CDR-L1 derived from L1-1 clone
KSSHSLLASGNQNNYLA

SEQ ID NO: 30 - protein, 17 aa, Artificial Sequence
  Synthetic CDR-L1 derived from L1-3 clone
KSSRSLLSSGNHKNYLA

SEQ ID NO: 31 - protein, 17 aa, Artificial Sequence
  Synthetic CDR-L1 derived from L1-4 clone
KSSKSLLASGNQNNYLA

SEQ ID NO: 32 - protein, 17 aa, Artificial Sequence
  Synthetic CDR-L1 derived from L1-12 clone
KSSRSLLASGNQNNYLA

SEQ ID NO: 33 - protein, 17 aa, Artificial Sequence
  Synthetic CDR-L1 derived from L1-22 clone
KSSHSLLASGNQNNYLA

SEQ ID NO: 34 - protein, 7 aa, Artificial Sequence
  Synthetic CDR-L2 derived from L2-9 clone
WASKRVS

SEQ ID NO: 35 - protein, 7 aa, Artificial Sequence
  Synthetic CDR-L2 derived from L2-12 clone
WGSTRVS

SEQ ID NO: 36 - protein, 7 aa, Artificial Sequence
  Synthetic CDR-L2 derived from L2-16 clone
WGSTRVP

SEQ ID NO: 37 - protein, 9 aa, Artificial Sequence
  Synthetic CDR-L3 derived from L3-32 clone
QQSYSKPFT

SEQ ID NO: 38 - DNA, 1416 bp, Artificial Sequence
  Synthetic nucleotide sequence of heavy chain of chAbF46
gaattcgccgccaccatggaatggagctgggtttttctcgtaacacttttaaatggtatccagtgtgaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatggacaccctgagagctgaggacagtgccacttattactgtgcaagagataactggtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 39 - DNA, 759 bp, Artificial Sequence
  Synthetic nucleotide sequence of light chain of chAbF46
gaattcactagtgattaattcgccgccaccatggattcacaggcccaggtcctcatgttgctgctgctatcggtatctggtacctgtggagacattttgatgacccagtctccatcctccctgactgtgtcagcaggagagaaggtcactatgagctgcaagtccagtcagagtcttttagctagtggcaaccaaaataactacttggcctggcaccagcagaaaccaggacgatctcctaaaatgctgataatttgggcatccactagggtatctggagtccctgatcgcttcataggcagtggatctgggacggatttcactctgaccatcaacagtgtgcaggctgaagatctggctgtttattactgtcagcagtcctacagcgctccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 40 - protein, 447 aa, Artificial Sequence
  Synthetic amino acid sequence of H1-heavy
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 447 aa, Artificial Sequence
  Synthetic amino acid sequence of H3-heavy
EVQLVESGGGLVQPGGSLRLSCAASGFTFTDYYMSWVRQAPGKGLEWLGFIRNKANGYTTEYSASVKGRFTISRDNSKNSLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 447 aa, Artificial Sequence
  Synthetic amino acid sequence of H4-heavy
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 220 aa, Artificial Sequence
  Synthetic amino acid sequence of H1-light
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 220 aa, Artificial Sequence
  Synthetic amino acid sequence of H2-light
DIVMTQTPLSLPVTPGEPASISCKSSQSLLASGNQNNYLAWHLQKPGQSPQMLIIWASTRVSGVPDRFSGSGSGTDFTLKISRVEAEDVGVYYCQQSYSAPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 220 aa, Artificial Sequence
  Synthetic amino acid sequence of H3-light
DIVMTQSPDSLAVSLGERATINCKSSQSLLASGNQNNYLAWYQQKPGQPPKLLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 219 aa, Artificial Sequence
  Synthetic amino acid sequence of H4-light
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 47 - DNA, 1350 bp, Artificial Sequence
  Synthetic nucleotide sequence of H1-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgaaaaccgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 48 - DNA, 1350 bp, Artificial Sequence
  Synthetic nucleotide sequence of H3-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgcgtgctgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 49 - DNA, 1350 bp, Artificial Sequence
  Synthetic nucleotide sequence of H4-heavy
gaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 50 - DNA, 669 bp, Artificial Sequence
  Synthetic nucleotide sequence of H1-light
gacatcgtgatgacccagtctccagactccctggctgtgtctctgggcgagagggccaccatcaactgcaagtccagccagagtcttttagctagcggcaaccaaaataactacttagcttggcaccagcagaaaccaggacagcctcctaagatgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 51 - DNA, 669 bp, Artificial Sequence
  Synthetic nucleotide sequence of H2-light
gatattgtgatgacccagactccactctccctgcccgtcacccctggagagccggcctccatctcctgcaagtccagtcagagtcttttagctagtggcaaccaaaataactacttggcctggcacctgcagaagccagggcagtctccacagatgctgatcatttgggcatccactagggtatctggagtcccagacaggttcagtggcagtgggtcaggcactgatttcacactgaaaatcagcagggtggaggctgaggatgttggagtttattactgccagcagtcctacagcgctccgctcacgttcggacagggtaccaagctggagctcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 52 - DNA, 669 bp, Artificial Sequence
  Synthetic nucleotide sequence of H3-light
gacatcgtgatgacccagtctccagactccctggctgtgtctctgggcgagagggccaccatcaactgcaagtccagccagagtcttttagctagcggcaaccaaaataactacttagcttggtaccagcagaaaccaggacagcctcctaagctgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 53 - DNA, 669 bp, Artificial Sequence
  Synthetic nucleotide sequence of H4-light
gatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagcgctccgctcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 54 - protein, 23 aa, Artificial Sequence
  Synthetic linker between VH and VL
GLGGLGGGGSGGGGSGGSSGVGS

SEQ ID NO: 55 - DNA, 1088 bp, Artificial Sequence
  Synthetic polynucleotide encoding scFv of huAbF46 antibody
gctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaac

SEQ ID NO: 56 - DNA, 5597 bp, Artificial Sequence
  Synthetic expression vector including polynucleotide encoding scFv of huAbF46 antibody
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagctaattctacttcatacattttcaattaagatgcagttacttcgctgtttttcaatattttctgttattgctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaacccgctgatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccgtgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaatttcttgaagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctggctagt

SEQ ID NO: 57 - protein, 13 aa, Artificial Sequence
  Synthetic U6-HC7 hinge
EPKSCDCHCPPCP

SEQ ID NO: 58 - DNA, 435 bp, Artificial Sequence
  Synthetic polynucleotide encoding CDR-L3 derived from L3-1 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacg

SEQ ID NO: 59 - DNA, 435 bp, Artificial Sequence
  Synthetic polynucleotide encoding CDR-L3 derived from L3-2 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgggcagtcctacagccgtccgctcacgttcggacagggtaccaaggtggagatcaaacgtacg

SEQ ID NO: 60 - DNA, 435 bp, Artificial Sequence
  Synthetic polynucleotide encoding CDR-L3 derived from L3-3 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgcacagtcctacagccatccgttctctttcggacagggtaccaaggtggagatcaaacgtacg

SEQ ID NO: 61 - DNA, 435 bp, Artificial Sequence
  Synthetic polynucleotide encoding CDR-L3 derived from L3-5 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtttacgttcggacagggtaccaaggtggagatcaaacgtacg

SEQ ID NO: 62 - protein, 462 aa, Artificial Sequence
  Synthetic polypeptide consisting of heavy chain of huAbF46-H4-A1, U6-HC7 hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1410 bp, Artificial Sequence
  Synthetic polynucleotide encoding polypeptide consisting of heavy chain of huAbF46-H4-A1, U6-HC7 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaaagctgcgattgccactgtcctccatgtccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 64 - protein, 461 aa, Artificial Sequence
  Synthetic polypeptide consisting of heavy chain of huAbF46-H4-A1, human IgG2 hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1407 bp, Artificial Sequence
  Synthetic polynucleotide encoding polypeptide consisting of heavy chain of huAbF46-H4-A1, human IgG2 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagaggaagtgctgtgtggagtgccccccctgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 66 - protein, 460 aa, Artificial Sequence
  Synthetic polypeptide consisting of heavy chain of huAbF46-H4-A1, human IgG2 hinge and constant region of human IgG2
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - DNA, 1404 bp, Artificial Sequence
  Synthetic polynucleotide encoding polypeptide consisting of heavy chain of huAbF46-H4-A1, human IgG2 hinge and constant region of human IgG2
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 68 - protein, 240 aa, Artificial Sequence [sequence omitted]

SEQ ID NO: 69 - DNA, 758 bp, Artificial Sequence
  Synthetic polynucleotide encoding polypeptide consisting of light chain of huAbF46-H4-A1(H36Y) and human kappa constant region
aattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggtaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 70 - protein, 240 aa, Artificial Sequence
  Synthetic polypeptide consisting of light chain of huAbF46-H4-A1 and human kappa constant region
MDSQAQVLMLLLLSVSGTCGDIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 19 aa, Artificial Sequence
  Synthetic epitope in SEMA domain of c-Met
FSPQIEEPSQCPDCVVSAL

SEQ ID NO: 72 - protein, 10 aa, Artificial Sequence
  Synthetic epitope in SEMA domain of c-Met
PQIEEPSQCP

SEQ ID NO: 73 - protein, 5 aa, Artificial Sequence
  Synthetic epitope in SEMA domain of c-Met
EEPSQ

SEQ ID NO: 74 - protein, 117 aa, Artificial Sequence
  Synthetic heavy chain variable region of anti-c-Met antibody (AbF46 or huAbF46-H1)
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSS

SEQ ID NO: 75 - protein, 114 aa, Artificial Sequence
  Synthetic light chain variable region of anti-c-Met antibody (AbF46 or huAbF46-H1)
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKR

SEQ ID NO: 76 - DNA, 1416 bp, Artificial Sequence; identical to SEQ ID NO: 38
  Synthetic nucleotide sequence of heavy chain of anti-c-Met antibody (AbF46 or huAbF46-H1)

SEQ ID NO: 77 - DNA, 759 bp, Artificial Sequence; identical to SEQ ID NO: 39
  Synthetic nucleotide sequence of light chain of anti-c-Met antibody (AbF46 or huAbF46-H1)

SEQ ID NO: 78 - DNA, 4170 bp, Artificial Sequence
  Synthetic polynucleotide encoding c-Met protein
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 79 - protein, 444 aa, Artificial Sequence
  Synthetic SEMA domain of c-Met
LHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNG

SEQ ID NO: 80 - protein, 451 aa, Artificial Sequence
  Synthetic PSI-IPT domain of c-Met
YTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTG

SEQ ID NO: 81 - protein, 313 aa, Artificial Sequence
  Synthetic TyrKc domain of c-Met
VHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 82 - DNA, 1332 bp, Artificial Sequence
  Synthetic polynucleotide encoding SEMA domain of c-Met
ctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggc

SEQ ID NO: 83 - DNA, 1299 bp, Artificial Sequence
  Synthetic polynucleotide encoding PSI-IPT domain of c-Met
tacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacagga

SEQ ID NO: 84 - DNA, 939 bp, Artificial Sequence
  Synthetic polynucleotide encoding TyrKc domain of c-Met
gtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 85 - protein, 13 aa, Artificial Sequence
  Synthetic heavy chain CDR3 of anti-c-Met antibody
DNWFAYWGQGTLV

SEQ ID NO: 86 - protein, 10 aa, Artificial Sequence
  Synthetic light chain CDR3 of anti-c-Met antibody
LTFGAGTKLE

SEQ ID NO: 87 - protein, 117 aa, Artificial Sequence
  Synthetic heavy chain variable region of monoclonal antibody AbF46
EVKLVESGGGLVQPGGSLRLSCATSGFTFTDYYMSWVRQPPGKALEWLGFIRNKANGYTTEYSASVKGRFTISRDNSQSILYLQMDTLRAEDSATYYCARDNWFAYWGQGTLVTVSA

SEQ ID NO: 88 - protein, 114 aa, Artificial Sequence
  Synthetic light chain variable region of anti-c-Met antibody
DILMTQSPSSLTVSAGEKVTMSCKSSQSLLASGNQNNYLAWHQQKPGRSPKMLIIWASTRVSGVPDRFIGSGSGTDFTLTINSVQAEDLAVYYCQQSYSAPLTFGAGTKLELKR

SEQ ID NO: 89 - protein, 17 aa, Artificial Sequence
  Synthetic light chain CDR3 of anti-c-Met antibody
QQSYSAPLTFGAGTKLE

SEQ ID NO: 90 - protein, 117 aa, Artificial Sequence
  Synthetic heavy chain variable region of AT-VH1
EVKLVESGGGLVQPGGSLRLSCATSGFTFTDYYMSWVRQPPGKGLEWLGFIRNKANGYTTEYSASVKGRFTISRDNSKSTLYLQMNSLRAEDSATYYCARDNWFAYWGQGTLVTVSS

SEQ ID NO: 91 - protein, 117 aa, Artificial Sequence
  Synthetic heavy chain variable region of AT-VH2
EVKLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 92 - protein, 117 aa, Artificial Sequence
  Synthetic heavy chain variable region of AT-VH3
EVQLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 93 - protein, 117 aa, Artificial Sequence
  Synthetic heavy chain variable region of AT-VH4
EVQLVESGGGLVQPGGSLRLSCATSGFTFTDYYMSWVRQPPGKGLEWLGFIRNKANGYTTEYSASVKGRFTISRDNSKNTLYLQMNSLRAEDTATYYCARDNWFAYWGQGTLVTVSS

SEQ ID NO: 94 - protein, 117 aa, Artificial Sequence
  Synthetic heavy chain variable region of AT-VH5
EVQLVESGGGLVQPGGSLRLSCATSGFTFTDYYMSWVRQPPGKGLEWLGFIRNKANGYTTEYSASVKGRFTISRDNSKNTLYLQMNSLRAEDTAVYYCARDNWFAYWGQGTLVTVSS

SEQ ID NO: 95 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKR

SEQ ID NO: 96 - protein, 113 aa, Artificial Sequence
  Synthetic light chain variable region of AT-Vk1
DILMTQSPSSLTASVGDRVTMTCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 97 - protein, 113 aa, Artificial Sequence
  Synthetic light chain variable region of AT-Vk2
DILMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 98 - protein, 113 aa, Artificial Sequence
  Synthetic light chain variable region of AT-Vk3
DIQMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 99 - protein, 113 aa, Artificial Sequence
  Synthetic light chain variable region of AT-Vk4
DIQMTQSPSSLSASVGDRVTITCKSSQSLLASGNQNNYLAWHQQKPGKAPKMLIIWASTRVSGVPDRFSGSGSGTDFTLTISSLQAEDVAVYYCQQSYSAPLTFGQGTKLEIK

SEQ ID NO: 100 - protein, 13 aa, Artificial Sequence
  Synthetic modified hinge region(U7-HC6)
EPSCDKHCCPPCP

SEQ ID NO: 101 - protein, 13 aa, Artificial Sequence
  Synthetic modified hinge region(U6-HC7)
EPKSCDCHCPPCP

SEQ ID NO: 102 - protein, 12 aa, Artificial Sequence
  Synthetic modified hinge region(U3-HC9)
ERKCCVECPPCP

SEQ ID NO: 103 - protein, 14 aa, Artificial Sequence
  Synthetic modified hinge region(U6-HC8)
EPRDCGCKPCPPCP

SEQ ID NO: 104 - protein, 13 aa, Artificial Sequence
  Synthetic modified hinge region(U8-HC5)
EKCDKTHTCPPCP

SEQ ID NO: 105 - protein, 15 aa, Artificial Sequence
  Synthetic human hinge region
EPKSCDKTHTCPPCP

SEQ ID NO: 106 - protein, 17 aa, Artificial Sequence
  Synthetic CDR-L1 of antibody L3-11Y
KSSQSLLAWGNQNNYLA

SEQ ID NO: 107 - protein, 114 aa, Artificial Sequence
  Synthetic amino acid sequence of light chain variable region of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKR

SEQ ID NO: 108 - protein, 220 aa, Artificial Sequence
  Synthetic amino acid sequence of light chain of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - DNA, 115 bp, Artificial Sequence
  Synthetic IGLV-1L1 Intron
gtgacaggatggggaccaagaaaggggccctgggaagcccatggggccctgctttctcctcttgtctccttttgtctcttgtcaatcaccatgtctgtgtctctctcacttccag

SEQ ID NO: 110 - DNA, 206 bp, Artificial Sequence
  Synthetic IGKV Intron
gtgagaatatttagaaaaagctaaaactaattctttgaaccattaattttcttaattaggaacctggcaccatatggaacttggcttgtttttaaatgtgatttttttttaagtaatgcgtattctttcatcttgtgctactagattagtggtgatttcattaagcagatgcttatattgtgctaatgtttgctgtatgttttcag

SEQ ID NO: 111 - DNA, 82 bp, Artificial Sequence
  Synthetic IGH Intron
gtgagtgtctcagggatccagacatgggggtatgggaggtgcctctgatcccagggctcactgtgggtctctctgttcacag

SEQ ID NO: 112 - DNA, 133 bp, Artificial Sequence
  Synthetic Chimeric Intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 113 - DNA, 963 bp, Artificial Sequence
  Synthetic Intron A
gtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcacc

SEQ ID NO: 114 - DNA, 20 bp, Artificial Sequence
  Synthetic Overlapping region with huCMV
gtctatataagcagagctct

SEQ ID NO: 115 - DNA, 30 bp, Artificial Sequence
  Synthetic Restriction enzyme site (EcoRI/NheI/XhoI)
gaattcgaaggagctagcggagaactcgag

SEQ ID NO: 116 - DNA, 1848 bp, Artificial Sequence
  Synthetic human CMV immediate-early enhancer/promoter
ctgcagtgaataataaaatgtgtgtttgtccgaaatacgcgtttgagatttctgtcccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagttgatttttgggcatacgcgatatctggcgatacgcttatatcgtttacgggggatggcgatagacgcctttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgcacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccagcgaatctcgggtacgtgttccggacatggggctcttctccggtagcggcggagcttctacatccagccctgctcccatcctcccactcatggtcctcggcagctccttgctcctaacagtggaggccagacttaggcacagcacgatgcccaccaccaccagtgtgcccacaaggccgtggcggtagggtatgtgtctgaaaatgagctc

SEQ ID NO: 117 - DNA, 795 bp, Artificial Sequence
  Synthetic hCMV promoter fragment
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcag

SEQ ID NO: 118 - DNA, 594 bp, Artificial Sequence
  Synthetic hCMV promoter fragment
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaa

SEQ ID NO: 119 - DNA, 509 bp, Artificial Sequence
  Synthetic hCMV promoter fragment
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 120 - DNA, 508 bp, Artificial Sequence
  Synthetic hCMV promoter fragment
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 121 - DNA, 584 bp, Artificial Sequence
  Synthetic hCMV promoter fragment
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 122 - DNA, 588 bp, Artificial Sequence
  Synthetic hCMV promoter fragment
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctc

SEQ ID NO: 123 - DNA, 680 bp, Artificial Sequence
  Synthetic hCMV promoter fragment
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactcta

SEQ ID NO: 124 - DNA, 597 bp, Artificial Sequence
  Synthetic hCMV promoter fragment
gaccgccatgttgacattgattattgactagttattaatagtaatcaattacggggtcattagttcatagcccatatatggagttccgcgttacataacttacggtaaatggcccgcctcgtgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccggccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctc

SEQ ID NO: 125 - DNA, 53 bp, Artificial Sequence
  Synthetic Nucleotide sequence further added to hCMV fragment
actagaagctttattgcggtagtttatcacagttaaattgctaacgcagtcag

SEQ ID NO: 126 - DNA, 7 bp, Artificial Sequence
  Synthetic Nucleotide sequence further added to hCMV fragment
gactcta

SEQ ID NO: 127 - DNA, 25 bp, Artificial Sequence
  Synthetic CA-Fw primer
taacagggtaatatagacgcgtgga

SEQ ID NO: 128 - DNA, 20 bp, Artificial Sequence
  Synthetic hCMV-Rv primer
caattgagagctctgcttat

SEQ ID NO: 129 - DNA, 23 bp, Artificial Sequence
  Synthetic LInt-Fw primer
agagctctcaattggtgacagga

SEQ ID NO: 130 - DNA, 24 bp, Artificial Sequence
  Synthetic KInt-Fw primer
agagctctcaattggtgagaatat

SEQ ID NO: 131 - DNA, 24 bp, Artificial Sequence
  Synthetic HInt-Fw primer
agagctctcaattggtgagtgtct

SEQ ID NO: 132 - DNA, 23 bp, Artificial Sequence
  Synthetic CInt-Fw primer
agagctctcaattggtaagtatc

SEQ ID NO: 133 - DNA, 24 bp, Artificial Sequence
  Synthetic CA-Rev primer
ttctcgagttctccgctagctcct

SEQ ID NO: 134 - protein, 114 aa, Artificial Sequence
  Synthetic light chain variable region of anti c-Met antibody
DIQMTQSPSSLSASVGDRVTITCKSSQSLLASGNQNNYLAWYQQKPGKAPKMLIIWASTRVSGVPSRFSGSGSGTDFTLTISSLQPEDFATYYCQQSYSRPYTFGQGTKVEIKR